(19) United States
(12) Patent Application Publication
(10) Pub. No.: US 2019/0060463 A1
(43) Pub. Date: Feb. 28, 2019

(54) BRIDGED POLYETHYLENE GLYCOL-ALIPHATIC POLYESTER BLOCK COPOLYMER, PREPARATION METHOD FOR SAME, INTERMEDIATE OF SAME, AND USES THEREOF

(71) Applicant: University Of Science And Technology Of China, Hefei, Anhui (CN)

(72) Inventors: Jun Wang, Hefei, Anhui (CN); Chunyang Sun, Hefei, Anhui (CN); Congfei Xu, Hefei, Anhui (CN); Zhiting Cao, Hefei, Anhui (CN); Hongjun Li, Hefei, Anhui (CN)

(73) Assignee: University Of Science And Technology Of China, Hefei, Anhui (CN)

(21) Appl. No.: 15/772,980

(22) PCT Filed: Nov. 3, 2015
(86) PCT No.: PCT/CN2015/093676

§ 371 Date: Aug. 13, 2018

(51) Int. Cl. A61K 47/34; C08G 65/333; C08G 65/332; C08G 81/00; A61K 9/14; A61K 31/7105; A61P 35/00

(52) U.S. Cl. CPC A61K 47/34; C08G 65/33306; C08G 65/3328; A61P 35/00; A61K 9/146; A61K 31/7105; C08G 81/00

(57) ABSTRACT

This invention relates to a bridged polyethylene glycol-aliphatic polyester block copolymer, which can be used in the production of delivery carriers for micromolecular chemotherapeutic drugs and nucleic acid drugs. Nanoscale pharmaceutical carriers produced from the bridged block polymer may be subjected to specific degradation under pH environments in tumor tissues or tumor cells so as to change the structure of nanoparticles, enhance cell uptake or intracellular drug release, and improve the sensitivity of tumor cells to drugs.

### TECHNICAL FIELD

This invention relates to the field of pharmaceutical carriers, and particularly to the field of pharmaceutical carriers comprising bridged polyethylene glycol-aliphatic polyester block copolymers.

### BACKGROUND ART

Nanoscale pharmaceutical carriers can protect drug molecules, change distribution in vivo and pharmacokinetics of drug molecules, increase intracellular concentration of drugs, and enhance druggability of candidate drugs, thereby significantly enhancing pharmaceutical efficacy and reducing toxic and side effects. A large number of drug adjuvant materials based on amphipathic block polymers as well as their formulations have arisen in fields of basic research and application, great economic benefits are also obtained with respect to polymer-based nano-drugs which have been approved. Among these, representative amphipathic block polymers are polyethylene glycol-aliphatic polyester block polymers having excellent biodegradability, bioabsorbability, and biocompatibility, which are mainly polyethylene glycol-polylactide, polyethylene glycol-polyglycolic acid, and polyethylene glycol-polycaprolactone.

In the early stage of researches in nano-drugs, it is typically considered that the distribution in vivo of drugs may be improved and the object of enhancing the therapeutic effects may be achieved by elongating the circulation in vivo of particles;however, it has been found in subsequent researches that drug-loading nanoparticles should break through multiple barriers in vivo so as to effectively improve the therapeutic effects of drugs in the treatment of tumors. These barriers include that: 1) suitable and elongated circulation time in the blood is required for molecules or particles of drugs; 2) the enrichment of drugs in tumor tissues is required to be enhanced for loading drugs by nanoparticles; 3) the uptake of drugs by tumor cells is required to be improved; and 4) rapid release of drug molecules from tumor cells is required. Covering the surface of a nano-drug, which is formed from a conventional amphipathic block polymer, for example the aforementioned polyethylene glycol-aliphatic polyester block polymer, and a drug molecule, with a hydrophilic component such as polyethylene glycol (PEG), contributes to the elongation of circulation time in vivo of the drug and promotes the enrichment of the drug in tumor tissues. However, PEG molecules covering the surface of the nano-drug hinder the uptake of the nano-drug by tumor cells, or even hinder the release of drug molecules from nanoparticles entering into cells, which limits the drug delivery performance of these amphipathic polymers as pharmaceutical carriers and restricts conversion and application thereof.

Using special physicochemical microenvironments in tumor cells, researchers have designed an amphipathic block polymer “bridged” by a sensitive chemical bond, typically such as an amphipathic polymer “bridged” by a disulfide bond or a diselenide bond. This “bridged” amphipathic polymer refers to a kind of amphipathic polymer in which a hydrophilic component (such as PEG) and other hydrophobic components (such as aliphatic polyester) are “bridged” by using a special chemical bond. Generally, a “bridged” chemical bond is sensitive to special environments (such as pH and reducing environments) and can be rapidly degraded under specific environments, so that hydrophilic and hydrophobic components are separated. Finally, the composition of the nanoparticle produced therefrom is changed, leading to the change in properties of the nanoparticle or the damage of the structure of the nanoparticle. Consequently, the escape of the particle from the endosome is enhanced or the release of the drug is promoted. These “bridged” amphipathic blocks, which are sensitive to special physicochemical microenvironments in tumor cells may solve a part of problems, such as the problem with respect to the intracellular release of drugs from nanoscale pharmaceutical carriers, but have no effect in the uptake of nanoscale pharmaceutical carriers by tumor cells.

As the development of bridged polymers and the increasing expansion of “chemical bond libraries”, relevant work is gradually shifting to how to change the component of the bridging chemical bond to achieve extracellular degradation of bridged polymers, thereby overcoming the impediment to the cell uptake of nanoparticles due to polyethylene glycol. Tumor tissue matrix specific microenvironments outside tumor cells are mainly divided into weakly acidic environments (pH 6.5-7.0) caused by the Warburg effect and specifically expressed enzymatic substances relevant to tumorigenesis and progression. The latter is more easily used to design relevant bridged polymers. For example, a bridged polyethylene glycol-nonameric arginine-polycaprolactone block polymer (PEG-XPLG*LAGR₉X-PCL) is formed after respectively bonding polyethylene glycol and polycaprolactone to terminals of a polypeptide (XPLG*LAGR₉X) sensitive to matrix metalloprotease 2 (MMP2). However, the effects of practical applications of polymer carriers, which are polyethylene glycol-aliphatic polyester block copolymers bridged by chemical bonds sensitive to enzymes, are undesirable. The pharmaceutical efficacy of carrier systems lacks reproducibility and universality. Additionally, the synthesis processes of these bridged polymers typically have low reaction efficiencies and poor reproducibility, and lack the implementability in expanding synthesis.

The environment in a solid tumor exhibits to be weakly acidic (pH 6.5-7.0), and after entering a tumor cell by endocytosis, nanoparticles will further undergo more strongly acidic environments in endosomes/lysosomes (pH 5.0-5.5). The speed of bonding of a hydrogen ion to chemical bond is far higher, compared to the bonding of an enzyme to a polypeptide chemical bond. The application range of constructing a bridged polyethylene glycol-aliphatic polyester block polymer with a chemical bond having a pH responsiveness will be more practical and wider. However, since there is a relatively small difference between pH values in and outside tumor cells described above and pH values in normal physiological environments, harsher requirements for the design and the response sensitivity of a “bridging” chemical bond are proposed. Therefore, this invention focuses on intending to provide a polyethylene glycol-aliphatic polyester block copolymer block polymer bridged by an amide bond responsive to pH in tumor matrices and cells, used as a delivery carrier for micromolecular chemotherapeutic drugs and nucleic acid drugs. It can be specifically degraded under pH environments in tumor matrices and cells to change the structure of nanoparticles, enhance the uptake of nanoparticles by tumor cells, and increase the amounts of drugs in cells, thereby finally improving therapeutic effects of drugs.

### SUMMARY OF THE INVENTION

In order to solve the above technical problems, this invention first provides:

In the first aspect, a bridged polyethylene glycol-aliphatic polyester block copolymer, having the following general structural formula III:

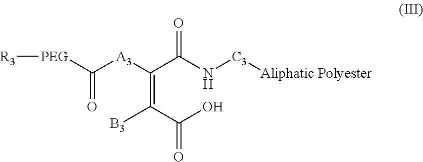

wherein A₃ is selected from C_{g}Hₕ, and g and h are integers, 0≤g≤4, and 0≤h≤10; B₃ is a methyl group or absent; C₃ is selected from CᵢHⱼ, and i and j are integers, 1≤i≤20, and 2≤j≤42; R₃ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue, and aliphatic polyester represents an aliphatic polyester residue.

Here, A₃ is preferably absent or an alkylene group having a carbon atom number of 1-4;

C₃ is preferably an alkylene group having a carbon atom number of 1-20, more preferably an alkylene group having a carbon atom number of 1-6; and

R₃ is preferably an alkyl group having a carbon atom number of 1-6, an alkoxy group having a carbon atom number of 1-6, an aryl group having a carbon atom number of 6-20, an aryloxy group having a carbon atom number of 6-20, or a halogen atom, and the alkyl group, the alkyloxy group, the aryl group, and the aryloxy group may be further substituted, and more preferably R₃ is an alkyloxy group having a carbon atom number of 1-6.

2. The bridged polyethylene glycol-aliphatic polyester block copolymer according to the first aspect described above, wherein the polyethylene glycol residue is represented by the following general formula:

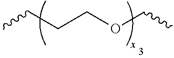

wherein x₃ is an integer and 1≤x₃≤500.

The aliphatic polyester residue is a residue of poly(ε-caprolactone), polylactide, or poly(lactic-co-glycolic acid).

Here, the number average molecular weight of the aliphatic polyester is preferably 2000-20000; more preferably 5000-15000.

Here, the ratio of repeating unit numbers of lactic acid to glycolic acid in the poly(lactic-co-glycolic acid) is preferably 10-90/90-10, more preferably 20-80/80-20, and further preferably 75/25.

3. A production method of the bridged polyethylene glycol-aliphatic polyester block copolymer according to the first or second aspect described above, comprising: performing ring opening polymerization reaction of an aliphatic polyester monomer by using a maleamidic acid derivative modified polyethylene glycol as an initiator to obtain a bridged polyethylene glycol-aliphatic polyester block copolymer; or performing macromolecular coupling reaction between a maleamidic acid derivative modified polyethylene glycol and an aliphatic polyester having an amino terminal group to obtain a bridged polyethylene glycol-aliphatic polyester block copolymer.

Here, the ring opening polymerization reaction is preferably performed under an anhydrous condition;

the reaction is preferably performed in the presence of a catalyst;

the catalyst is preferably an organic heterocyclic molecule, 1,5,7-triazabicyclo[4.4.01]dec-5-ene;

the solvent is preferably dichloromethane;

the reaction is preferably performed at 0° C.;

the reaction time is preferably 10-120min; and

the resultant crude product is preferably subjected to purification treatment, for example precipitation treatment. 4. A fourth aspect of this invention provides a maleamidic acid derivative modified polyethylene glycol, having the following general structural formula II:

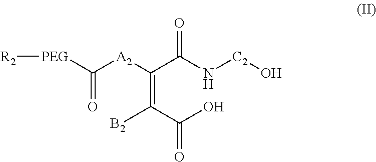

wherein A₂ is selected from C_{c}H_{d}, and c and d are integers, 0≤c≤4, and 0≤d≤10; B₂ is a methyl group or absent; C₂ is selected from CₑH_{f}, and e and f are integers, 1≤e≤20, and, 2≤f≤42; R₂ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue.

Here, A₂, B₂, C₂, R₂, and PEG may be the same as A₃, B₃, C₃, R₃, and PEG in the bridged polyethylene glycol-aliphatic polyester block copolymer according to the first aspect, and the scopes may also be preferably the same.

5. A fifth aspect of this invention provides a production method of the maleamidic acid derivative modified polyethylene glycol of the fourth aspect, comprising mixing an amino alcohol with a polyethylene glycol containing a maleic anhydride group at the terminal, and forming an amide bond by performing ring opening reaction using a primary amine group in the amino alcohol and the maleic anhydride group to obtain a maleamidic acid derivative modified polyethylene glycol.

Here, the reaction is preferably performed in an anhydrous solution system or under an anhydrous condition;

the reaction is preferably performed at room temperature;

the crude product is preferably subjected to purification treatment; and

the purification treatment preferably comprises liquid separation by extraction and precipitation.

6. A sixth aspect of this invention provides a polyethylene glycol containing a maleic anhydride group at the terminal, having the following general structural formula I:

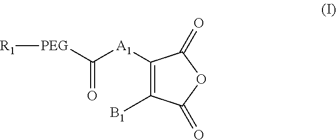

wherein A₁ is selected from CₐH_{b}, and a and b are integers, 0≤a≤4, and 0≤b≤10; B₁ is a methyl group or absent; R₁ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue.

Here, A₁, B₁, R₁, and PEG may be the same as A₃, B₃, R₃, and PEG in the bridged polyethylene glycol-aliphatic polyester block copolymer according to the first aspect, and the scopes may also be preferably the same.

7. A seventh aspect of this invention provides a production method of the polyethylene glycol containing a maleic anhydride group at the terminal of the sixth aspect, comprising subjecting a carboxy group in a maleic anhydride substituent to acyl chlorination and then to reaction with a terminal hydroxy group of the polyethylene glycol.

The agent for acyl chlorination is preferably oxalyl dichloride or dichlorosulfane;

the solvent is preferably anhydrous dichloromethane;

the reaction temperature is preferably 0-40° C.;

the crude product is preferably subjected to a purification treatment; and

the purification treatment preferably comprises extraction and precipitation.

8. An eighth aspect of this invention provides a pharmaceutical carrier or nucleic acid carrier produced from the bridged polyethylene glycol-aliphatic polyester block copolymer of the first or second aspect.

Here, the production method of the carrier preferably comprises dissolving a bridged block copolymer formed from poly(ε-caprolactone), polylactide, or poly(lactic-co-glycolic acid) with polyethylene glycol in a water-insoluble organic phase and performing emulsification with water under an ultrasonic condition (for example, 0° C., 50-200 W, 30-120 s) so as to produce a nanoparticle; and at the meanwhile, if a hydrophobic drug is added to the organic phase, the entrapment of the drug may be achieved;

the organic phase is preferably dichloromethane, chloroform, or ethyl acetate; and

the hydrophobic drug is preferably one or more of taxol, docetaxel, de-hydrochlorinated Adriamycin, all-trans retinoic acid, and hydroxy camptothecin.

9. A ninth aspect of this invention provides a drug loaded nanoparticle or nucleic acid loaded nanoparticle produced from the pharmaceutical carrier of the eighth aspect.

Here, the production method of the nanoparticle preferably comprises dissolving a cationic lipid and a bridged block copolymer formed from poly(ε-caprolactone), polylactide, or poly(lactic-co-glycolic acid) with polyethylene glycol in a water-insoluble organic phase, performing primary emulsification with an aqueous siRNA solution (for example, 0° C., 50-200 W, 30-120 s), then performing secondary emulsification with water (for example, 0° C., 50-200 W, 30-120 s), and removing the organic phase to obtain a highly efficiently siRNA-entrapping nanoparticle.

The organic phase is preferably dichloromethane, chloroform, or ethyl acetate; and

the cationic lipid may be preferably N,N-bihydroxyethyl-N-methyl-N-2-(cholesteryloxycarbonyl amino)ethyl ammonium bromide and trimethyl-2,3-dioleoyloxypropylammonium bromide.

10. A tenth aspect of this invention provides use of the pharmaceutical carrier or nucleic acid carrier produced from the maleamidic acid derivative modified polyethylene glycol of the fourth aspect, the pharmaceutical carrier or nucleic acid carrier produced from the polyethylene glycol containing a maleic anhydride group at the terminal of sixth aspect, the pharmaceutical carrier or nucleic acid carrier of the eighth aspect, or the drug loaded nanoparticle or nucleic acid loaded nanoparticle of the ninth aspect in the manufacture of an anti-tumor drug.

### DESCRIPTION OF DRAWINGS

FIG. 1. The chemical structure and synthetic route of a PEG derivative terminated with methyl maleic anhydride in Example 1 of this invention.

FIG. 2. The characterization of a PEG derivative terminated with methyl maleic anhydride by hydrogen nuclear magnetic resonance spectra in Example 1 of this invention, wherein the solvent is deuterated chloroform. FIG. 3. The characterization of a PEG derivative terminated with methyl maleic anhydride by carbon nuclear magnetic resonance spectra in Example 1 of this invention, wherein the solvent is deuterated chloroform. FIG. 4. The characterization of PEG derivatives terminated with methyl maleic anhydride having different molecular weights by hydrogen nuclear magnetic resonance spectra in Example 1 of this invention, wherein the solvent is deuterated chloroform.

FIG. 5. The chemical structure and synthetic route of a PEG derivative terminated with maleic anhydride in Example 2 of this invention. FIG. 6. The characterization of a PEG derivative terminated with maleic anhydride by hydrogen nuclear magnetic resonance spectra in Example 2 of this invention, wherein the solvent is deuterated chloroform. FIG. 7. The characterization of a PEG derivative terminated with maleic anhydride by carbon nuclear magnetic resonance spectra in Example 2 of this invention, wherein the solvent is deuterated chloroform. FIG. 8. The characterization of PEG derivatives terminated with maleic anhydride having different molecular weights by hydrogen nuclear magnetic resonance spectra in Example 2 of this invention, wherein the solvent is deuterated chloroform.

FIG. 9. The chemical structure and synthetic route of α-PEG-P-methyl-6-hydroxyhexyl maleamidic acid in Example 3 of this invention. FIG. 10. The characterization of α-PEG-β-methyl-6-hydroxyhexyl maleamidic acid by hydrogen nuclear magnetic resonance spectra in Example 3 of this invention, wherein the solvent is deuterated chloroform. FIG. 11. The characterization of α-PEG-β-methyl-6-hydroxyhexyl maleamidic acid by carbon nuclear magnetic resonance spectra in Example 3 of this invention, wherein the solvent is deuterated chloroform. FIG. 12. The characterization of α-PEG-β-methyl-6-hydroxyhexyl maleamidic acids having different molecular weights by hydrogen nuclear magnetic resonance spectra in Example 3 of this invention, wherein the solvent is deuterated chloroform.

FIG. 13. The chemical structure and synthetic route of α-PEG-6-hydroxyhexyl maleamidic acid in Example 4 of this invention. FIG. 14. The characterization of α-PEG-6-hydroxyhexyl maleamidic acid by hydrogen nuclear magnetic resonance spectra in Example 4 of this invention, wherein the solvent is deuterated chloroform. FIG. 15. The characterization of α-PEG-6-hydroxyhexyl maleamidic acid by carbon nuclear magnetic resonance spectra in Example 4 of this invention, wherein the solvent is deuterated chloroform. FIG. 16. The characterization of α-PEG-β-methyl-6-hydroxyhexyl maleamidic acids having different molecular weights by hydrogen nuclear magnetic resonance spectra in Example 4 of this invention, wherein the agent is deuterated chloroform.

FIG. 17. The chemical structure and synthetic route of a polyethylene glycol-Dlinkₘ-polylactide copolymer bridged by an acid-catalytically hydrolyzable amide bond Dlinkₘ in Example 5 of this invention. FIG. 18. The characterization of a Dlinkₘ bridged polyethylene glycol-Dlinkₘ-polylactide copolymer by gel permeation chromatography in Example 5 of this invention. FIG. 19. The characterization of the chemical structure of a Dlinkₘ bridged polyethylene glycol-Dlinkₘ-polylactide copolymer by hydrogen nuclear magnetic resonance spectra in Example 5 of this invention, wherein the solvent is deuterated chloroform. FIG. 20. The characterization of the chemical structure of a Dlinkₘ bridged polyethylene glycol-Dlinkₘpolylactide copolymer by carbon nuclear magnetic resonance spectra in Example 5 of this invention, wherein the solvent is deuterated chloroform.

FIG. 21. The chemical structure and synthetic route of a polyethylene glycol-Dlink-polylactide copolymer bridged by an acid-catalytically hydrolyzable amide bond Dlink in Example 6 of this invention. FIG. 22. The characterization of a Dlink bridged polyethylene glycol-Dlink-polylactide copolymer by gel permeation chromatography in Example 6 of this invention.

FIG. 23. The characterization of the chemical structure of a polyethylene glycol-Dlink-polylactide copolymer bridged by an acid-catalytically hydrolyzable amide bond Dlink by hydrogen nuclear magnetic resonance spectra in Example 6 of this invention, wherein the agent is deuterated chloroform. FIG. 24. The characterization of the chemical structure of a Dlink bridged polyethylene glycol-Dlink-polylactide copolymer by carbon nuclear magnetic resonance spectra in Example 6 of this invention, wherein the solvent is deuterated chloroform.

FIG. 25. The chemical structure and synthetic route of a polyethylene glycol-Dlink-polycaprolactone (or polyethylene glycol-Dlinkₘ-polycaprolactone) copolymer bridged by an acid-catalytically hydrolyzable amide bond Dlink (or Dlinkₘ) in Example 7 of this invention. FIG. 26. The characterization of a Dlinkₘ bridged polyethylene glycol-Dlinkₘ-polycaprolactone copolymer by gel permeation chromatography in Example 7 of this invention. FIG. 27. The characterization of a Dlink bridged polyethylene glycol-Dlink-polycaprolactone copolymer by gel permeation chromatography in Example 7 of this invention. FIG. 28. The characterization of the chemical structure of a polyethylene glycol-Dlinkₘpolycaprolactone copolymer bridged by an acid-catalytically hydrolyzable amide bond Dlinkₘ by hydrogen nuclear magnetic resonance spectra in Example 7 of this invention, wherein the solvent is deuterated chloroform. FIG. 29. The characterization of the chemical structure of a polyethylene glycol-Dlink-polycaprolactone copolymer bridged by an acid-catalytically hydrolyzable amide bond Dlink by hydrogen nuclear magnetic resonance spectra in Example 7 of this invention, wherein the agent is deuterated chloroform. FIG. 30. The characterization of the chemical structure of a Dlinkₘ bridged polyethylene glycol-Dlinkₘ-polycaprolactone copolymer by carbon nuclear magnetic resonance spectra in Example 7 of this invention, wherein the agent is deuterated chloroform. FIG. 31. The characterization of the chemical structure of a Dlink bridged polyethylene glycol-Dlink-polycaprolactone copolymer by carbon nuclear magnetic resonance spectra in Example 7 of this invention, wherein the agent is deuterated chloroform.

FIG. 32. The chemical structure and synthetic route of a polyethylene glycol-poly(lactic-co-glycolic acid) copolymer bridged by acid-catalytically hydrolyzable amide bonds Dlinkₘ and Dlink in Example 8 of this invention. FIG. 33. The characterization of a Dlinkₘ bridged polyethylene glycol-Dlinkₘ-poly(lactic-co-glycolic acid) copolymer by gel permeation chromatography in Example 8 of this invention. The subscripts at each PLGA represent the polymerization degrees of D,L-LA and GA, respectively. FIG. 34. The characterization of a Dlink bridged polyethylene glycol-Dlink-poly(lactic-co-glycolic acid) copolymer by gel permeation chromatography in Example 8 of this invention. The subscripts at each PLGA represent the polymerization degrees of D,L-LA and GA, respectively. FIG. 35. The characterization of the chemical structure of a polyethylene glycol-Dlinkₘ-poly(lactic-co-glycolic acid) copolymer bridged by an acid-catalytically hydrolyzable amide bond Dlinkₘ by hydrogen nuclear magnetic resonance spectra in Example 8 of this invention, wherein the agent is deuterated chloroform. FIG. 36. The characterization of the chemical structure of a polyethylene glycol-Dlink-poly(lactic-co-glycolic acid) copolymer bridged by an acid-catalytically hydrolyzable amide bond Dlink by hydrogen nuclear magnetic resonance spectra in Example 8 of this invention, wherein the agent is deuterated chloroform. FIG. 37. The characterization of the chemical structure of a Dlinkₘ bridged polyethylene glycol-Dlinkₘ-poly(lactic-co-glycolic acid) copolymer by carbon nuclear magnetic resonance spectra in Example 8 of this invention, wherein the agent is deuterated chloroform. FIG. 38. The characterization of the chemical structure of a Dlink bridged polyethylene glycol-Dlink-poly(lactic-co-glycolic acid) copolymer by carbon nuclear magnetic resonance spectra in Example 8 of this invention, wherein the agent is deuterated chloroform.

FIG. 39. (A) Schematic diagram of the degradation of a bridged polymer; Detections by high-performance liquid chromatography for degradation behaviors of nanoparticles formed by assembling (B) mPEG₁₁₃-Dlinkₘ-PDLLA₄₂, (C) mPEG₁₁₃-Dlinkₘ-PDLLA₇₁, (D) mPEG₁₁₃-Dlinkₘ-PDLLA₁₄₂, and (E) mPEG₁₁₃-Dlink-PDLLA₁₄₀ under different pH environments, in Example 10 of this invention.

FIG. 40. Detections by flow cytometry for cell uptake of MDA-MB-231 cells for NP_{PDLLA} and Dₘ-NP_{PDLLA} treated under different pH conditions in Example 11 of this invention. The production methods of fluorescently labeled Dₘ-NP_{PDLLA} and NP_{PDLLA} are as described in Example 9, and the components are mPEG₁₁₃-Dlinkₘ-PDLLA₁₄₂ and mPEG₁₁₃-b-PDLLA₁₄₀.

FIG. 41. Circulation in vivo of nanoparticles of different components in the body of an ICR mouse in Example 12 of this invention. The production methods of RhoB labeled nanoparticles Dₘ-NP_{PDLLA} and NP_{PDLLA} are as described in Example 9, and the components are mPEG₁₁₃-Dlinkₘ-PDLLA₁₄₂ and mPEG₁₁₃-b-PDLLA₁₄₀.

FIG. 42. Inhibitions of MDA-MB-231 in situ tumor models in different treatment groups in Example 13 of this invention, wherein the administration dosage of docetaxel is 3.5 mg/kg. The production methods of NP_{PDLLA/DTXL}, Dₘ-NP_{PDLLA/DTXL}, and D-NP_{PDLLA/DTXL}, which entrap docetaxel, are as described in Example 9, and the components are mPEG₁₁₃-b-PDLLA₇₂, mPEG₁₁₃-Dlinkₘ-PDLLA₇₀, and mPEG₁₁₃-Dlink-PDLLA₇₅, respectively. The significant difference is calculated by a function of t-test, *p<0.05.

FIG. 43. Release of siRNAs by double-emulsified nanoparticles carrying siRNAs under different pH conditions in Example 14 of this invention. The production methods of nanoparticles Dₘ-NP_{PLGA//FAM-siNC} and NP_{PLGA/FAM-siNC}, which carry siRNAs, are as described in Example 9, and the components are mPEG₁₁₃-Dlinkₘ-PLGA_{161/54} and mPEG₁₁₃-b-PLGA_{165/56}.

FIG. 44. Detections by flow cytometry for uptake behaviors of MDA-MB-231 cells for double-emulsified nanoparticles carrying siRNAs treated under different pH conditions in Example 15 of this invention. The production methods of nanoparticles Dₘ-NP_{PLGA/Cy5-siNC} and NP_{PLGA/Cy5-siNC}, which carry siRNAs, are as described in Example 9, and the components are mPEG₁₁₃-Dlinkₘ-PLGA_{161/54} and mPEG₁₁₃-b-PLGA_{165/56}. MFI is the mean fluorescence intensity in cells. The significant difference is calculated by a function of t-test, **p<0.01. FIG. 45. Detections by high-performance liquid chromatography for uptake behaviors of MDA-MB-231 cells for double-emulsified nanoparticles carrying siRNAs treated under different pH conditions in Example 15 of this invention. The production methods of nanoparticles Dₘ-NP_{PLGA/Cy5-siNC} and NP_{PLGA/Cy5-siNC}, which carry siRNAs, are as described in Example 9, and the components are mPEG₁₁₃-Dlinkₘ-PLGA_{161/54} and mPEG₁₁₃-b-PLGA_{165/56}. The significant difference is calculated by a function of t-test, *p<0.05. FIG. 46. Detections by laser confocal scanning microscope observation for uptake behaviors of MDA-MB-231 cells for double-emulsified nanoparticles carrying siRNAs treated under different pH conditions in Example 15 of this invention. The production methods of nanoparticles Dₘ-NP_{PLGA/Cy5-siNC} and NP_{PLGA/Cy5-siNC, which carry siRNAs, are as described in Example} 9, and the components are mPEG₁₁₃-Dlinkₘ-PLGA_{161/54} and mPEG₁₁₃-b-PLGA_{165/56}.

FIG. 47. Downregulation of the PLK₁ gene in MDA-MB-231 cells by double-emulsified nanoparticles carrying siRNAs under conditions of pH 7.4 (A) and pH 6.5 (B) in Example 16 of this invention. The production methods of nanoparticles Dₘ-NP_{PLGA/Cy5-siNC} NP_{PLGA/Cy5-siNC}, Dₘ-NP_{PLGA/siPLK1}, and NP_{PLGA/siPLK1}, which carry siRNAs, are as described in Example 9, and the components are mPEG₁₁₃-Dlinkₘ-PLGA_{161/54} and mPEG₁₁₃-b-PLGA_{165/56}. The significant difference is calculated by a function of t-test, *p<0.05.

FIG. 48. Effects of double-emulsified nanoparticles carrying siRNAs (Dₘ-NP_{PLGA/Cy5-siNC}, NP_{PLGA/Cy5-siNC}, Dₘ-NP_{PLGA/siPLK1}, and NP_{PLGA/siPLK1}) on the PLK1 protein in MDA-MB-231 cells under a condition of pH 6.5 detected by a Western blotting method in Example 17 of this invention.

FIG. 49. Effects of double-emulsified nanoparticles carrying siRNAs on the cell viability of MDA-MB-231 cells under a condition of pH 6.5 in Example 18 of this invention. The production methods of nanoparticles (Dₘ-NP_{PLGA/Cy5-siNC}, NP_{PLGA/Cy5-siNC}, Dₘ-NP_{PLGA/siPLK1}, and NP_{PLGA/siPLK1}), which carry siRNAs, are as described in Example 9, and the components are mPEG₁₁₃-Dlinkₘ-PLGA_{161/54} and mPEG₁₁₃-b-PLGA_{165/56}. The significant difference is calculated by a function of t-test, *p<0.05.

FIG. 50. Distribution in vivo of double-emulsified nanoparticles which carry siRNAs in the body of a MDA-MB-231 tumor-bearing mouse in Example 19 of this invention. The production methods of nanoparticles (Dₘ-NP_{PLGA/Cy5-siNC} and NP_{PLGA/Cy5-siNC}), which carry siRNAs, are as described in Example 9, and the components are mPEG₁₁₃-Dlinkₘ-PLGA_{161/54} and mPEG₁₁₃-b-PLGA_{165/56}. The significant difference is calculated by a function of t-test, **p<0.01.

FIG. 51. Inhibitions of MDA-MB-231 in situ tumor models by different treatment groups in Example 20 of this invention. The production methods of nanoparticles (Dₘ-NP_{PLGA/siPLK1} and NP_{PLGA/siPLK}₁), which carry siRNAs, are as described in Example 9, and the components are mPEG₁₁₃-Dlinkₘ-PLGA_{161/54} and mPEG₁₁₃-b-PLGA_{165/56}. The significant difference is calculated by a function of t-test, *p<0.05.

### DESCRIPTION OF EMBODIMENTS

This invention first provides a polyethylene glycol (PEG) derivative containing a maleic anhydride group at the terminal, and the polyethylene glycol derivative involved in this invention has the following general structural formula I:

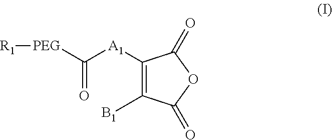

wherein A₁ may be selected from CₐH_{b}, and a and b are integers, 0≤a≤4, and 0≤b≤10; B₁ may be a methyl group or absent; R₁ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue.

Here, A₁ is preferably absent or an alkylene group having a carbon atom number of 1-4;

R₁ is preferably an alkyl group having a carbon atom number of 1-6, an alkoxy group having a carbon atom number of 1-6, an aryl group having a carbon atom number of 6-20, an aryloxy group having a carbon atom number of 6-20, or a halogen atom, and the alkyl group, the alkyloxy group, the aryl group, and the aryloxy group may be further substituted, and more preferably R₁ is an alkyloxy group having a carbon atom number of 1-6.

The polyethylene glycol PEG is represented by the following general formula:

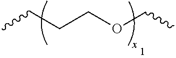

wherein xi is an integer and 20≤x₁500.

This invention further provides a synthesis method of a polyethylene glycol derivative containing a maleic anhydride group at the terminal.

The synthesis method of the polyethylene glycol derivative containing a maleic anhydride group at the terminal comprises: subjecting a carboxy group in a maleic anhydride substituent to acyl chlorination to produce an acyl-chlorinated substituent of maleic anhydride, performing reaction with a terminal hydroxy group of the polyethylene glycol under a mild condition, and performing purification in a manner of extraction and precipitation, so as to finally synthesize a polyethylene glycol derivative containing a maleic anhydride group at the terminal. In the method, the agent for acyl chlorination is oxalyl dichloride, dichlorosulfane, or the like, but is not limited to this scope only; the solvent selected is anhydrous dichloromethane, and the reaction temperature is 0-40° C.

Next, this invention provides another maleamidic acid derivative modified polyethylene glycol (PEG), having the following general structural formula II:

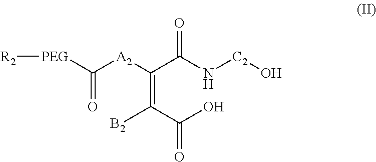

wherein A₂ is selected from C_{c}H_{d}, and c and d are integers, 0≤c≤4, and 0≤d≤10; B₂ is a methyl group or absent; C₂ is selected from CₑH_{f}, and e and f are integers, 1≤e≤20, and, 2≤f≤42; R₂ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue.

Here, A₂ is preferably absent or an alkylene group having a carbon atom number of 1-4;

C₂ is preferably an alkylene group having a carbon atom number of 1-20, more preferably an alkylene group having a carbon atom number of 1-6; and

R₂ is preferably an alkyl group having a carbon atom number of 1-6, an alkoxy group having a carbon atom number of 1-6, an aryl group having a carbon atom number of 6-20, an aryloxy group having a carbon atom number of 6-20, or a halogen atom, and the alkyl group, the alkyloxy group, the aryl group, and the aryloxy group may be further substituted, and more preferably R₂ is an alkyloxy group having a carbon atom number of 1-6.

The polyethylene glycol is represented by the following general formula:

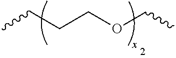

wherein x₂ is an integer and 20≤x₂≤500.

This invention provides a corresponding synthesis method of the polyethylene glycol derivative described above. The synthesis method of the polyethylene glycol derivative comprises mixing an amino alcohol with a polyethylene glycol derivative containing a maleic anhydride group at the terminal in a certain ratio in a mild anhydrous solution system, forming a specific amide bond at room temperature by performing ring opening reaction using a primary amine group in the amino alcohol and the maleic anhydride group, and performing treatment and purification on the product after reaction in a manner of liquid separation by extraction and precipitation, so as to obtain a final expected product.

This invention also provides a bridged polyethylene glycol-aliphatic polyester block copolymer, having the following general structural formula III:

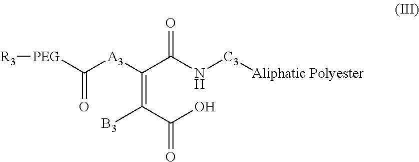

wherein A₃ is selected from C_{g}Hₕ, and g and h are integers, 0≤g≤4, and 0≤h≤10; B₃ is a methyl group or absent; C₃ is selected from CᵢHⱼ, and i and j are integers, 1≤i≤20, and 2≤j≤42; R₃ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue, and aliphatic polyester represents an aliphatic polyester residue.

Here, A₃ is preferably absent or an alkylene group having a carbon atom number of 1-4;

C₃ is preferably an alkylene group having a carbon atom number of 1-20, more preferably an alkylene group having a carbon atom number of 1-6; and

R₃ is preferably an alkyl group having a carbon atom number of 1-6, an alkoxy group having a carbon atom number of 1-6, an aryl group having a carbon atom number of 6-20, an aryloxy group having a carbon atom number of 6-20, or a halogen atom, and the alkyl group, the alkyloxy group, the aryl group, and the aryloxy group may be further substituted, and more preferably R₃ is an alkyloxy group having a carbon atom number of 1-6.

Here, the polyethylene glycol residue is represented by the following general formula:

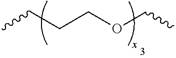

wherein x₃ is an integer and 1≤x₃≤500.

This invention provides a preferable synthesis method of a bridged polyethylene glycol-aliphatic polyester.

The preferable synthesis method of the bridged polyethylene glycol-aliphatic polyester comprises performing solution polymerization at 0° C. by using a polyethylene glycol derivative represented by the general formula II as a macromolecular initiator, an organic heterocyclic molecule 1,5,7-triazabicyclo[4.4.01]dec-5-ene as a catalyst under an anhydrous condition, and dichloromethane as a solvent to initiate ring opening polymerization reaction of monomers such as ε-caprolactone, lactide, glycolide, or the like, wherein the reaction time is 10-120 min, and performing purification in a manner of precipitation or the like, so as to finally synthesize a correspond bridged polyethylene glycol-aliphatic polyester. Unlike macromolecular coupling methods used in conventional synthesis of bridged polymers, this synthetic route is simple and controllable, which is favorable to reproduction; the product does not contain unreacted homopolymers, which facilitates purification and is more feasible.

Taking the following image as an example, a bridged polyethylene glycol-aliphatic polyester comprises an amide group having a specific structure compared to a non-bridged block polymer.

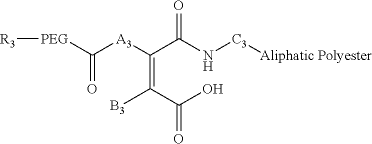

This allows that the bridged polymer involved in this invention has other characteristics. That is, specific degradation of the structure of the amide bond will occur under a weakly acidic condition to produce two different components, compared to a neutral condition.

When B₃ or B₂ is absent, the bridging amide bond may be degraded in a pH range of 5.0-6.0, wherein the speed of degradation is faster in pH range of 5.0-5.5:

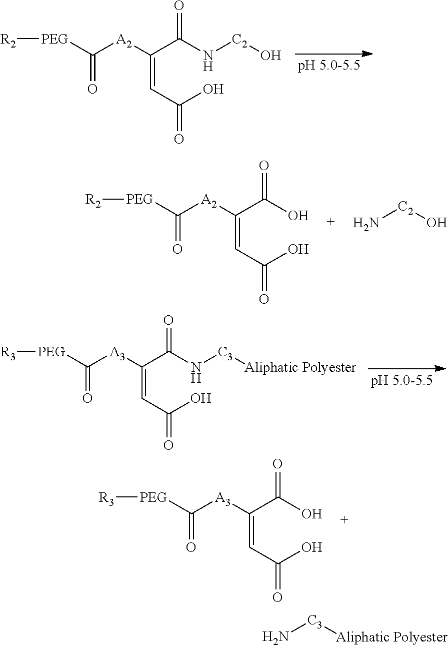

When B₃ or B₂ is a methyl group, the bridging amide bond may be degraded in a pH range of 6.0-7.0, wherein the speed of degradation is faster in pH range of 6.0-6.5:

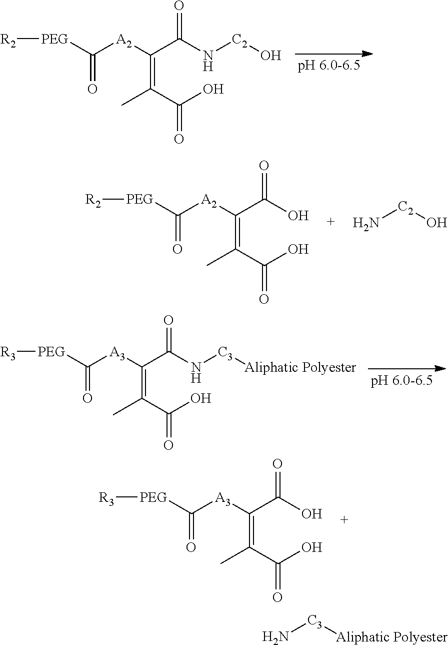

This invention also provides a method of forming a nano-drug delivery system by producing a block copolymer into a nanoparticle in water to carry a hydrophobic drug.

The production method of this invention preferably comprises dissolving a bridged block copolymer formed from poly(ε-caprolactone), polylactide, or poly(lactic-co-glycolic acid) with polyethylene glycol in a water-insoluble organic phase and performing emulsification with water under an ultrasonic condition (0° C., 50-200 W, 30-120 s) so as to produce a nanoparticle; and at the meanwhile, if a hydrophobic drug is added to the organic phase, the entrapment of the drug may be achieved. The efficiency of entrapment is stable and the reproducibility is good. The organic phase is dichloromethane, chloroform, or ethyl acetate, but is not limited to this scope; the hydrophobic drug is one or more of taxol, docetaxel, de-hydrochlorinated Adriamycin, all-trans retinoic acid, hydroxy camptothecin, and the like, but is not limited to this scope.

This invention provides a method of forming a nano-drug delivery system by producing a block copolymer into a nanoparticle in water to carry a hydrophilic small interfering RNA (siRNA).

The synthesis method comprises dissolving a cationic lipid and a bridged block copolymer formed from poly(ε-caprolactone), polylactide, or poly(lactic-co-glycolic acid) with polyethylene glycol in a water-insoluble organic phase, performing primary emulsification with an aqueous siRNA solution (0° C., 50-200 W, 30-120 s), then performing secondary emulsification with water (0° C., 50-200 W, 30-120 s), and removing the organic phase to obtain a highly efficiently siRNA-entrapping nanoparticle. The organic phase is dichloromethane, chloroform, or ethyl acetate, but is not limited to this scope; the cationic lipid may be N,N-bihydroxyethyl-N-methyl-N-2-(cholesteryloxycarbonylamino)ethyl ammonium bromide and trimethyl-2,3-dioleoyloxypropylammonium bromide, but is not limited to this scope.

With respect to the bridged block copolymer formed from poly(ε-caprolactone), polylactide, or poly(lactic-co-glycolic acid) with polyethylene glycol of this invention, the stability of its polyethylene glycol block is related to the pH condition of the environment. By using this property, the nanocarrier produced in this invention may be used in drug delivery in vivo for tumor tissues. In the process of blood circulation (pH 7.4, PEG stably present), it is possible to better elongate the time of circulation carrying drugs, improve the bioavailability of drugs, and reduce the toxicity in vivo of drugs, by the protective effect of polyethylene glycol on nanoparticles; whereas after the nanoparticle enters a tumor tissue (pH 6.0-6.5) or a tumor cell (pH 5.0-5.5), the bridging amide bond is responsively degraded,

PEG will be detached from the surface of the nanoparticle, the structure of the nanocarrier is damaged, and the capability of cell uptake or drug release is significantly improved. Therefore, multiple barriers faced by a conventional non-bridged polymer in the process of drug delivery are overcome, the content of active molecules in tumor cells is increased, and the stronger inhibition of tumor cell proliferation is achieved. Therefore, compared to free drugs widely used in current fields of clinical or basic researches, the bridged polymer of this invention may be desirable to improve its therapeutic effects and reduce toxic and side effects.

Additionally, the properties of the bridged polymer involved in this invention may be adjusted by adjusting the components of the polymer and the molecular weights of hydrophilic and hydrophobic blocks, and raw materials for reaction are easily available, reaction conditions are mild, and the process is simple, which are favorable to expansion and batch production.

A polyethylene glycol-aliphatic polyester block copolymer bridged by a chemical bond responsive to a specific pH in a tumor tissue or a tumor cell is obtained in this invention, and may be used in the entrapment of micromolecular drugs or macromolecular nucleic acid drugs and delivery in vivo thereof. Compared to a conventional polyethylene glycol-aliphatic polyester block polymer, the bridged polymer designed in this invention has the same properties in terms of particle stability, drug release in vitro, blood circulation, and the like, but is capable of regulating the degree of PEG on the surface of the nanoparticle by means of a specific pH in a tumor tissue or cell and enhancing cell uptake and intracellular drug release, thereby further improving therapeutic effects of drugs. In order to synthesize a bridged polyethylene glycol-aliphatic polyester block copolymer in this invention, polymerization reaction conditions used are mild, sources are easily available, and the process of purification after reaction is simple. After a nanoparticle is formed by assembling this polymer, the speed of response to tumor microenvironments is high and anti-tumor pharmaceutical efficacy can be significantly improved.

Certain specific embodiments of this invention are described by way of Examples below. However, these Examples are for illustrative purposes only and are not used to limit the scope of this invention.

### EXAMPLES

Abbreviations in Examples:

(1) mPEG, polyethylene glycol monomethyl ether

(2) PEG, polyethylene glycol

(3) CDM, 2-carboxyethyl-3 -methyl maleic anhydride

(4) CSM, 2-carboxyethyl-maleic anhydride

(5) TBD, 1,5,7-triazabicyclo[4.4.01]dec-5-ene

(6) ε-CL, ε-caprolactone

(7) PCL, poly

(ε-caprolactone)

(8) D,L-LA, racemic lactide

(9) PDLLA, polylactide

(10) GA, glycolide

(11) PLGA, poly

(lactic-co-glycolic acid) random copolymer

(12) BHEM-Chol, N,N-bihydroxyethyl-N-methyl-N-2-(cholesteryloxycarbonylamino)ethyl ammonium bromide

(13) RhoB, Rhodamine B

(14) DIC, N,N-diisopropylcarbodiimide

(15) DMAP, 4-dimethylaminopyridine

(16) PCL-RhoB, Rhodamine B labeled polycaprolactone

(17) DTXL, docetaxel

(18) siRNA, small interfering RNA

Sources of raw materials and treatment methods in Examples:

(1) mPEGs having molecular weights of 2000, 5000, 10000, and 20000 were available from Sigma-Aldrich Corporation, and water were removed by azeotropic distillation with toluene before use.

(2) mPEG having a molecular weight of 3400 was available from Shanghai Jingyu Biotech Co., Ltd., and water was removed by azeotropic distillation with toluene before use.

(3) PEG having a molecular weight of 6000 was available from Sigma-Aldrich Corporation, and water was removed by azeotropic distillation with toluene before use.

(4) D,L-LA was available from Jinan Daigang Biomaterial Co., Ltd.

(5) DTXL was available from Wuhan Dahua Weiye Pharmaceutical Co., Ltd.

(6) GA was available from Jinan Daigang Biomaterial Co., Ltd.

(7) c-CL was available from Daicel Chemical Co., Ltd., Japan.

(8) TBD was available from Sigma-Aldrich Corporation.

(9) Dimethyl α-oxoglutarate was available from Sigma-Aldrich Corporation.

(10) Thiazolyl blue was available from Sigma-Aldrich Corporation.

(11) Taxotere® was available from Sanofi-Aventis.

(12) Red and green fluorescently labeled siRNA (Cy5-siNC and FAM-siNC) were available from Suzhou Ribo Life Science Co., Ltd., an antisense strand sequence: 5′ -ACGUGACACGUUCGGAGAAdTdT-3′.

(13) PLK1 siRNA was available from Suzhou Ribo Life Science Co., Ltd., an antisense strand sequence: 5′-UAAGGAGGGUGAUCUUCUUCAdTdT-3′.

(14) Dichloromethane and chloroform, liquid chromatographic grade, were available from Duksan Corporation and were treated by SPS800 solvent purification apparatus of Mbraun Corporation.

(15) MDA-MB-231 cells were available from ATCC Corporation.

(16) Dulbecco's Modified Eagle Medium (DMEM) complete medium was available from Invitrogen Corporation.

(17) ICR mice were available from Beijing HFK Bioscience Co., Ltd.

(18) BALB/c nude mice were available from Beijing HFK Bioscience Co., Ltd.

(19) All other agents were analytically pure grade agents commercially available from conventional chemical agent corporations and are directly used, unless particularly indicated.

(20) The specific synthesis process of the cationic lipid BHEM-Chol was as follows.

In a 500-mL flask, 2-bromoethylamine hydrobromate (17.4 g, 85.0 mmol) and cholesteryl chloroformate (34.7 g, 77.3 mmol) were dissolved in a chloroform solution at −30° C., and triethylamine (24 mL, 172 mmol) was then dropped to the solution described above. After the reaction was performed overnight at room temperature, washed with 1 M hydrochloric acid in a saturated sodium chloride solution (150 mL) three times, and washed with a saturated sodium chloride solution (150 mL) once . After the organic phase was dried over anhydrous magnesium sulfate, the organic solvent was removed under reduced pressure to obtain a crude product. The crude product was recrystallized from ethanol once and acetone once to give cholesteryl N-(2-bromoethyl) carbamate. The cholesteryl N-(2-bromoethyl) carbamate obtained (4.8 g, 7.8 mmol) and N-methyldiethanolamine (1.2 g, 9.7 mmol) were added to 50 mL of dried toluene, and refluxed overnight. The reaction solution was precipitated into a large amount of ethyl ether, the precipitate was collected after filtration and dried under vacuum, and the product was recrystallized in ethanol twice to obtain a white solid, i.e., BHEM-Chol.

(21) The specific synthesis process of polyethylene glycol-polylactide (mPEG-b-PDLLA) was as follows.

Polyethylene glycol monomethyl ether (mPEG₁₁₃, 1.0 g, 0.2 mmol) and racemic lactide (2.5 g, 17.4 mmol) were added to a dry round-bottomed flask in a glovebox and heated at 130° C. until both of them are melted, stannous isooctoate (12.2 mg, 0.03 mmol) was added under stirring, and reaction was continued for 2 h. A crude product was dissolved in dichloromethane, and was precipitated into cold anhydrous ethyl ether/methanol (4/1, v/v) twice. The precipitate was collected, and dried under vacuum to a constant weight to obtain a polyethylene glycol-polylactide block polymer.

This polymer was subjected to analyses by hydrogen nuclear magnetic resonance spectra and gel permeation chromatography, wherein the polymerization degree of lactic acid was 140 and the molecular weight distribution of the polymer was 1.14, and was denoted by mPEG113-b-PDLLA₁₄₀.

(22) The specific synthesis process of polyethylene glycol-poly(lactic-co-glycolic acid) (mPEG-b-PLGA) was as follows.

Polyethylene glycol monomethyl ether (mPEG₁₁₃, 1.0 g, 0.2 mmol), racemic lactide (2.5 g, 17.4 mmol), and glycolide (0.76 g, 6.6 mmol) were added to a dry round-bottomed flask in a glovebox and heated at 130° C. until both of them are melted, stannous isooctoate (24.1 mg, 0.06 mmol) was added under stirring, and reaction was continued for 2 h. A crude product was dissolved in dichloromethane, and was precipitated into cold anhydrous ethyl ether/methanol (4/1, v/v) twice. The precipitate was collected, and dried under vacuum to a constant weight to obtain a polyethylene glycol-polylactide block polymer.

This polymer was subjected to analyses by hydrogen nuclear magnetic resonance spectra and gel permeation chromatography, wherein the polymerization degree of lactic acid was 165, the polymerization degree of glycolic acid was 56, and the molecular weight distribution of the polymer was 1.12, and was denoted by mPEG113-b-PLGA_{165/56}.

(23) The specific processes of synthesis and characterization of poly(ε-caprolactone) (PCL) were as follows, with reference to a literature (Polymer, 2009, 50, 5048-5054).

ε-CL (0.92 g, 8 mmol) was weighed, and approximately 15 mL of toluene was added. After stirring for approximately 10 minutes, 85 μL of a toluene solution containing 0.188 mmol of Al(OⁱPr)₃ was added, reaction was kept at 25° C. for 1 hour, and acetic acid was added to terminate the reaction. Toluene in the reaction liquid was concentrated with a rotary evaporator, then precipitated into cold methanol, and filtered. A resultant polymer was dried under vacuum at 25° C. to a constant weight so as to obtain a polycaprolactone homopolymer. This polymer was subjected to analyses by hydrogen nuclear magnetic resonance spectra and gel permeation chromatography, wherein the polymerization degree of caprolactone was 30 and the molecular weight distribution was 1.06, and was denoted by PCL₃₀.

(24) The synthesis process of Rhodamine B labeled polycaprolactone (PCL-RhoB) was as follows.

PCL₃₀ (0.50 g, 0.14 mmol), RhoB (0.211 g, 0.42 mmol), DIC (0.055 g, 0.70 mmol), and DMAP (0.055 g, 0.70 mmol) were weighed and dissolved in 10 mL of N,N-dimethylformamide, and reaction was performed in dark at 25° C. for 48 hours. After completion of the reaction, dialysis was performed in N,N-dimethylformamide to remove unreacted RhoB, drying under vacuum to a constant weight so as to obtain PCL-RhoB.

(25) The specific processes of synthesis and characterization of 2-carboxyethyl-3-methyl maleic anhydride (CDM) were as follows, with reference to a literature (Angewandte Chemie International Edition, 2013, 52, 6218-6221).

NaH (0.720 g, 0.030 mol), which was rinsed with 50 mL of anhydrous tetrahydrofuran twice at a low temperature, was suspended in 60 mL of anhydrous tetrahydrofuran, and stirred in an ice bath. Triethyl 2-phosphonopropionate (8.568 g, 0.036 mol) was slowly dropped into the suspension, and dimethyl α-oxoglutarate (6.960 g, 0.040 mmol) was added until no hydrogen is further generated in the system. Reaction was performed in an ice bath for 0.5 hours, and 30 mL of a saturated NH₄Cl solution was then added to terminate the reaction. The product was extracted with 100 mL of anhydrous ethyl ether twice. The organic phase was collected, dried over anhydrous MgSO₄, concentrated, and separated and purified by chromatography with a 200-mesh silica gel column, wherein the developing solvent was anhydrous ethyl ether/n-hexane (v/v, 2/1). A substance with R_{f}=0.6 was collected, dried, and further dissolved in 80 mL of absolute ethanol, and a KOH solution (2.0 M, 80 mL) was added, with heating reflux for 1 h. The system was cooled to room temperature, hydrochloric acid (6.0 M) was added to adjust pH to 2.0. The organic phase was collected by extraction with 200 mL of ethyl acetate, dried, and distilled under reduced pressure to remove the solvent, ethyl acetate. A crude product was recrystallized from anhydrous ethyl ether to obtain CDM (3.892 g, with a yield of 54.6%).

CDM was subjected to analysis by electrospray ionization mass spectrometry. This substance had a theoretical molecular weight of 184.15, while m/z=185.12 detected was the signal peak of [M+H⁺, demonstrating that the structure of the product was consistent with what was expected.

(26) The specific processes of synthesis and characterization of 2-carboxyethyl maleic anhydride (CSM) were as follows.

NaH (0.720 g, 0.030 mol), which was rinsed with 50 mL of anhydrous tetrahydrofuran twice at a low temperature, was suspended in 60 mL of anhydrous tetrahydrofuran, and stirred in an ice bath. Triethyl phosphonoacetate (8.064 g, 0.036 mol) was slowly dropped into the suspension, and dimethyl α-oxoglutarate (6.960 g, 0.040 mmol) was added until no hydrogen is further generated in the system. Reaction was performed in an ice bath for 0.5 hours, and 30 mL of a saturated NH₄Cl solution was then added to terminate the reaction. The product was extracted with 100 mL of anhydrous ethyl ether twice. The organic phase was collected, dried over anhydrous MgSO₄, concentrated, and separated and purified by chromatography with a 200-mesh silica gel column, wherein the developing solvent was anhydrous ethyl ether/n-hexane (v/v, 2/1). A substance with Rᵣ=0.65 was collected, dried, and further dissolved in 80 mL of absolute ethanol, and a KOH solution (2.0 M, 80 mL) was added, with heating reflux for 1 h. The system was cooled to room temperature, hydrochloric acid (6.0 M) was added to adjust pH to 2.0. The organic phase was collected by extraction with 200 mL of ethyl acetate, dried, and distilled under reduced pressure to remove the solvent, ethyl acetate. A crude product was recrystallized from anhydrous ethyl ether to obtain CSM (3.496 g, with a yield of 52.80%).

CSM was subjected to analysis by electrospray ionization mass spectrometry. This substance had a theoretical molecular weight of 170.12, while m/z=171.24 detected was the signal peak of [M+H⁺, demonstrating that the structure of the product was consistent with what was expected.

### Example 1

### Synthesis of PEG Derivative Terminated with Methyl Maleic Anhydride

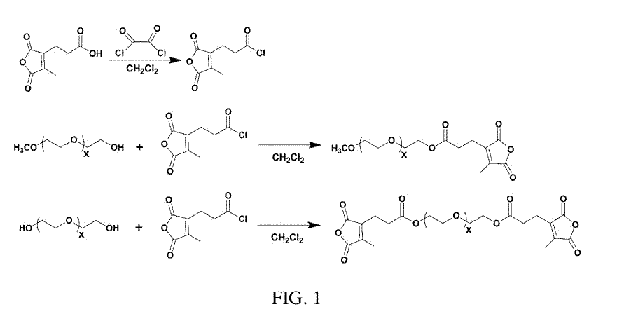

The chemical structure and synthetic route of a PEG derivative terminated with methyl maleic anhydride were as shown in FIG. 1.

CDM (1.840 g, 0.010 mol) was completely dissolved in anhydrous dichloromethane (20 mL) at 0° C., N,N-dimethylformamide (50 μL) and oxalyl dichloride (3.810 g, 0.030 mol) were successively added. After kept for 10 min, reaction was continued at 25° C. for 1 h. Dichloromethane was removed with a rotary evaporator, and N,N-dimethylformamide was removed by distillation under 15.0 Pa to obtain an intermediate, acyl-chlorinated CDM (1.96 g, with a yield of 97%).

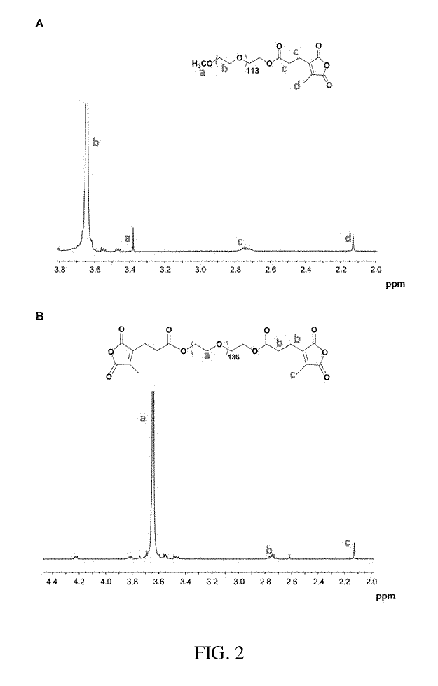

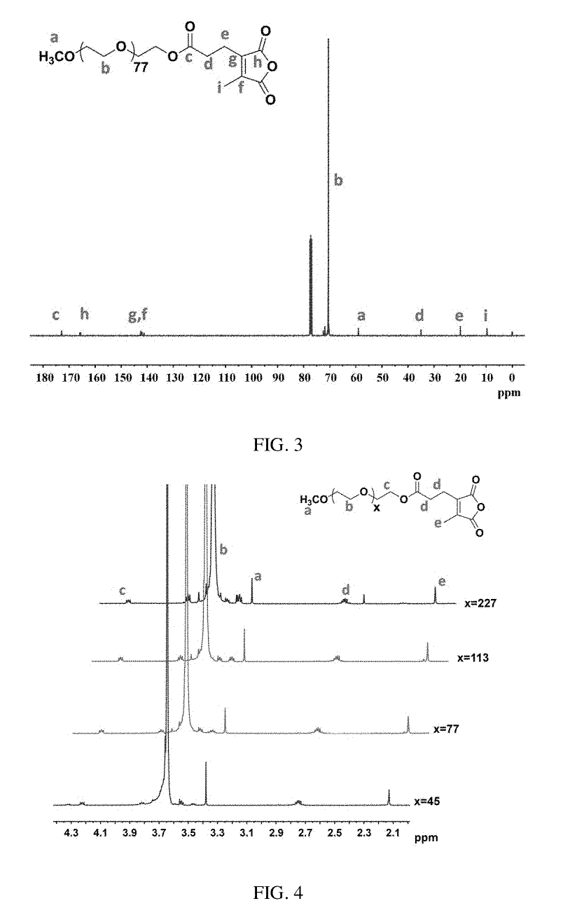

mPEG (or PEG), pyridine, and acyl-chlorinated CDM were added, in this order, to dried dichloromethane (the concentration of the polymer was 0.1 M) in a molar ratio of 1.0:6.0:3.0 and dissolved under stirring, subjected to reaction at 0° C. for 30 min, and transferred to 25° C. for further reaction for 2 h. After completion of the reaction, a saturated NH₄Cl solution having the same volume as that of CH₂Cl₂ was added, sufficient extraction was performed, and the organic phase was then collected. After dried over anhydrous MgSO₄, the organic phase was concentrated with a rotary evaporator and was precipitated with anhydrous ethyl ether at 0° C., and a solid was dried under vacuum to a constant weight. The PEG derivative terminated with methyl maleic anhydride synthesized as described above was subjected to hydrogen nuclear magnetic resonance spectrum (¹H NMR) analysis to measure its molecular structure, and the ¹H NMR spectra can be seen in FIG. 2. The PEG derivative terminated with methyl maleic anhydride described above was subjected to carbon nuclear magnetic resonance spectrum (¹³C NMR) analysis to further confirm its molecular structure, and ¹³C NMR can be seen in FIG. 3. PEG derivatives terminated with methyl maleic anhydride having different molecular weights were characterized by hydrogen nuclear magnetic resonance spectra, and the results can be seen in FIG. 4.

In FIG. 2 (A), the signal as shown by letter a was assigned to protonic hydrogen of a terminal methyl group in polyethylene glycol monomethyl ether, and the signal peak b at 3.67 ppm was assigned to protonic hydrogen of the backbone —CH₂CH₂O— of polyethylene glycol. Due to the bonding of CDM, c and d were both newly occurring signal peaks, wherein c was assigned to protonic hydrogen of two methylene groups in CDM; and d was assigned to protonic hydrogen of a methyl group in CDM. The bonding efficiency of CDM was obtained by calculating the integrated areas through the signal peak at 3.67 ppm and the signal peak at 2.13 ppm, and the reaction efficiency was higher than 98%.

In FIG. 2 (B), since a polyethylene glycol terminated with two hydroxy groups was used, the signal of protonic hydrogen of the terminal methyl group in polyethylene glycol monomethyl ether was absent, while other signals of protons were similar to those in FIG. 2 (A), and it was indicated by the calculation of integrated areas that the reaction efficiency of CDM was higher than 98%.

In FIG. 3, all carbon atoms in the PEG derivative terminated with methyl maleic anhydride can find respective signal assignments in ¹³C NMR. Among them, the signal peak near 70.4 ppm was assigned to carbon atoms in the backbone of polyethylene glycol, the signal peak at 174.4 ppm was a signal peak of a carbon atom in an ester bond generated in an acyl chlorination reaction, and the signal at 9.8 ppm was assigned to a carbon atom of a methyl group in an acid anhydride substituent. The results of ¹³C NMR further demonstrated the correctness of the structure of the PEG derivative terminated with methyl maleic anhydride produced.

¹H NMR spectra of products after the bonding of polyethylene glycol monomethyl ethers having different molecular weights to CDM can be seen from FIG. 4. Their signal peaks of protons were all similar to those in FIG. 2 (A), demonstrating that this method can synthesize PEG derivatives terminated with methyl maleic anhydride having different molecular weights.

### Example 2

### Synthesis of PEG Derivative Terminated with Maleic Anhydride

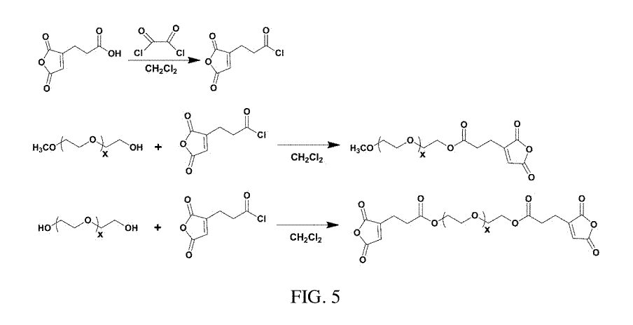

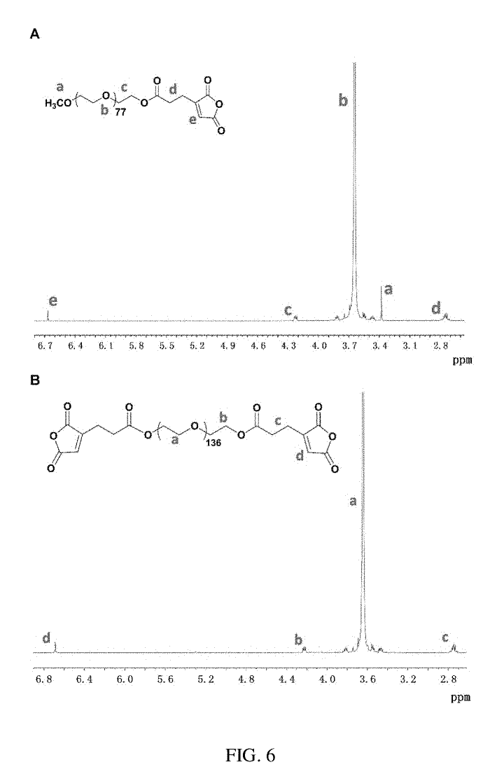

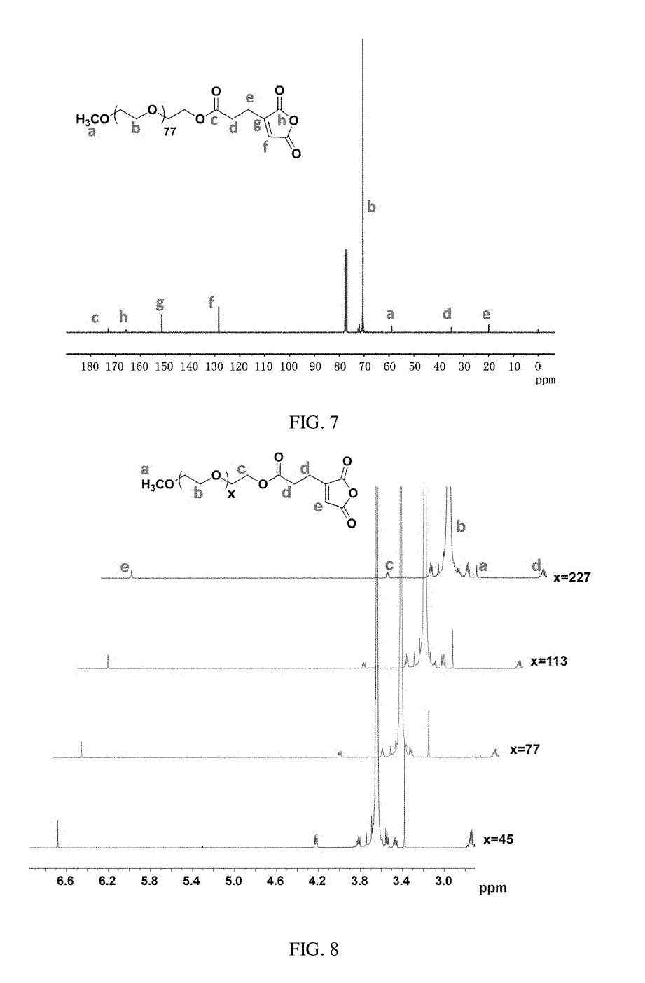

The chemical structure and synthetic route of a PEG derivative terminated with maleic anhydride were as shown in FIG. 5.

The specific synthesis method of a PEG derivative terminated with maleic anhydride was similar to that of the PEG derivative terminated with methyl maleic anhydride, and was performed by using 2-carboxyethyl maleic anhydride (CSM) instead of 2-carboxyethyl-3-methyl maleic anhydride (CDM).

CSM (3.080 g, 0.020 mol) was completely dissolved in anhydrous dichloromethane (35 mL) at 0° C., N,N-dimethylformamide (45 μL) and oxalyl dichloride (7.620 g, 0.060 mol) were successively added. After kept for 10 min, reaction was continued at 25° C. for 1 h. Dichloromethane was removed with a rotary evaporator, and N,N-dimethylformamide was removed by distillation under 15.0 Pa to obtain acyl-chlorinated CSM (3.420 g, with a yield of 91%).

mPEG (or PEG), pyridine, and acyl-chlorinated CSM were added, in this order, to dried dichloromethane (the concentration of mPEG or PEG was 0.1 M) in a molar ratio of 1.0:6.0:3.0 and dissolved under stirring, subjected to reaction at 0° C. for 30 min, and transferred to 25° C. for further reaction for 2 h. After completion of the reaction, a saturated NH₄Cl solution having the same volume as that of CH₂Cl₂ was added, sufficient extraction was performed, and the organic phase was then collected. After dried over anhydrous MgSO₄, the organic phase was concentrated with a rotary evaporator and was precipitated with anhydrous ethyl ether at 0° C., and a solid was dried under vacuum to a constant weight.

The PEG derivative terminated with maleic anhydride synthesized as described above was subjected to ¹H NMR analysis to measure its molecular structure, and the ¹H NMR spectra can be seen in FIG. 6. The PEG derivative terminated with maleic anhydride described above was subjected to ¹³C NMR analysis to further confirm its molecular structure, and ¹³C NMR can be seen in FIG. 7. PEG derivatives terminated with maleic anhydride having different molecular weights were characterized by hydrogen nuclear magnetic resonance spectra, and the results can be seen in FIG. 8.

In FIG. 6 (A), the signal peak as shown by letter a was assigned to protonic hydrogen of a terminal methyl group in polyethylene glycol monomethyl ether, and the single peak b at 3.65 ppm was assigned to protonic hydrogen of the backbone —CH₂CH₂O— of polyethylene glycol. Due to the bonding of CSM, c, d, and e were all newly occurring signal peaks, wherein d was assigned to protonic hydrogen of two methylene groups in CSM; and e was assigned to protons in maleic anhydride. The bonding efficiency of CSM was obtained by calculating the integrated areas through the signal peak at 3.65 ppm and the multiple peak at 2.74 ppm, and the reaction efficiency was higher than 97%.

In FIG. 6 (B), since a polyethylene glycol having two hydroxy groups was used, protonic hydrogen of the terminal methyl group in polyethylene glycol monomethyl ether was absent, while other signals of protons were consistent with those in FIG. 6 (A), and it was indicated by the calculation of integrated areas that the bonding efficiency of CSM was higher than 98%.

In FIG. 7, all carbon atoms in the PEG derivative terminated with maleic anhydride can find respective assignments in ¹³C NMR. Among them, the signal peak near 70.3 ppm was assigned to carbon atoms in the backbone of polyethylene glycol, the signal peak at 174.1 ppm was a signal peak of a carbon atom in an ester bond generated in an acyl chlorination reaction, and the signal at 9.8 ppm assigned to a carbon atom of a methyl group in an acid anhydride substituent of a CDM molecule that corresponds to the hydrogen nuclear magnetic resonance spectra is not present. The results of ¹³C NMR further demonstrated the correctness of the structure of the PEG derivative terminated with maleic anhydride produced.

¹H NMR spectra of products after the bonding of polyethylene glycol monomethyl ethers having different molecular weights to CSM can be seen from FIG. 8. Their signal peaks of protons were all similar to those in FIG. 6 (A), demonstrating that this method can synthesize PEG derivatives terminated with maleic anhydride having different molecular weights.

### Example 3

### Synthesis of α-PEG-β-Methyl-6-Hydroxyhexyl Maleamidic Acid

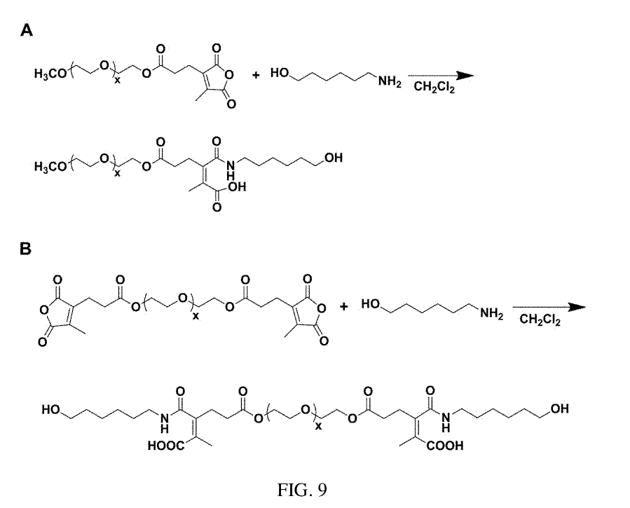

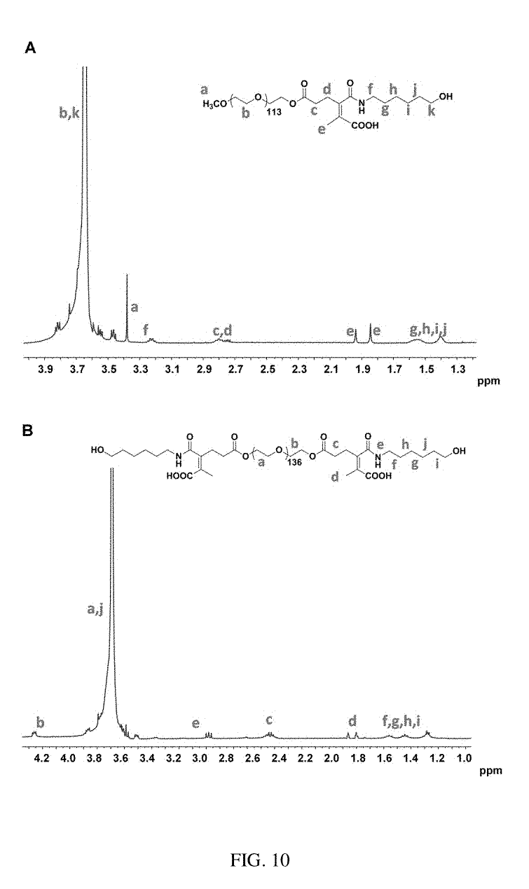

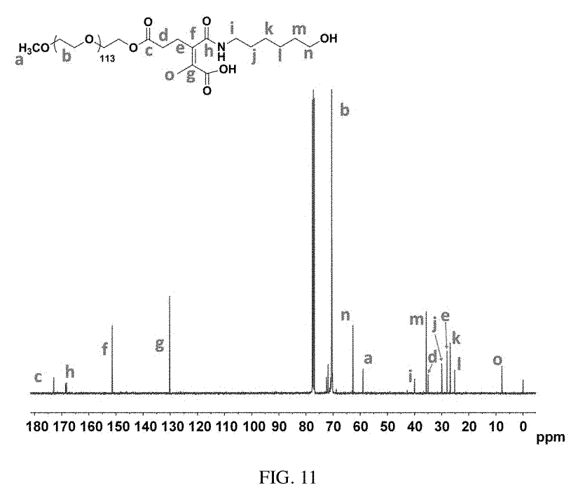

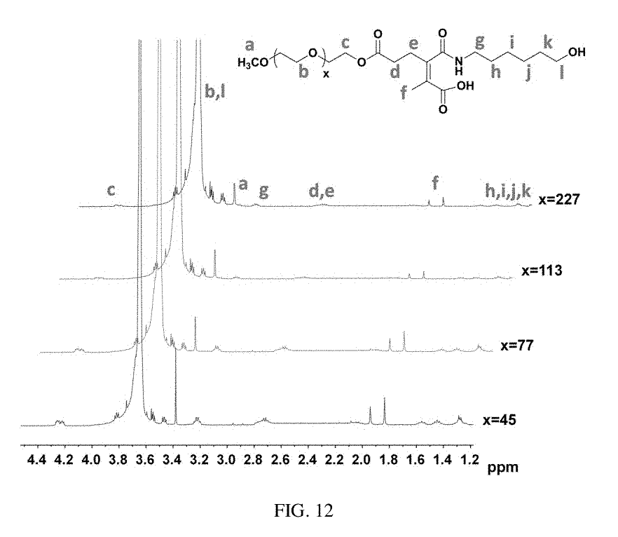

The chemical structure and synthetic route of α-PEG-β-methyl-6-hydroxyhexyl maleamidic acid were as shown in FIG. 9. The resultant product was represented by mPEG-Dlinkₘ-OH or HO-Dlinkₘ-PEG-Dlinkₘ-OH.

A PEG derivative terminated with methyl maleic anhydride and 6-amino-1-hexanol were completely dissolved in anhydrous CH₂Cl₂ together at 25° C., and reaction was performed under stirring (the concentration of the polymer was 0.1 M, the molar ratio of hydroxy groups of 6-amino-1-hexanol to polyethylene glycol was 3:1). After reaction for 12 h, a saturated NaCl solution was continuously added to extract twice. The organic phase was collected, and precipitated with excessive anhydrous ethyl ether at 0° C. After suction filtration under reduced pressure, a solid was dried under vacuum to a constant weight.

The α-PEG-β-methyl-6-hydroxyhexyl maleamidic acid synthesized as described above was subjected to ¹H NMR analysis to measure its molecular structure, and the ¹H NMR spectra can be seen in FIG. 10. The α-PEG-β-methyl-6-hydroxyhexyl maleamidic acid described above was subjected to ¹³C NMR analysis to further confirm its molecular structure, and ¹³C NMR can be seen in FIG. 11. α-PEG-β-methyl-6-hydroxyhexyl maleamidic acids having different molecular weights were characterized by hydrogen nuclear magnetic resonance spectra, and the results can be seen in FIG. 12.

In FIG. 10 (A), all the signal peaks in hydrogen nuclear magnetic resonance spectra were marked by letters a-k, which were sequentially assigned to respective proton signals of the corresponding product. Due to the introduction of 6-amino-1-hexanol, new proton signals were found at 3.23 ppm and 1.30-1.60 ppm and can be correctly assigned to protons of reacted 6-amino-1 -hexanol; while two signal peaks occurred at 1.85 ppm and 1.94 ppm instead of a single signal peak of a methyl group in an acid anhydride group of the original PEG derivative terminated with methyl maleic anhydride at 2.13 ppm due to the opening of the cyclic structure of acid anhydride. This demonstrated that ring opening reaction was successfully performed. The reaction efficiency of 6-amino-1 -hexanol and an acid anhydride group was obtained by calculating the integrated areas through the signal peak of a methyl group of polyethylene glycol monomethyl ether at 3.38 ppm and the multiple peak at 1.30-1.60 ppm, demonstrating the efficiency of the ring opening reaction was more than 96%.

In FIG. 10 (B), since a polyethylene glycol having two hydroxy groups was used, the signal of protonic hydrogen of the terminal methyl group in polyethylene glycol monomethyl ether was absent, while other signals of protons were consistent with those in FIG. 10 (A), and it was indicated by the calculation of integrated areas that the ring opening efficiency of maleic anhydride was higher than 96%.

In FIG. 11, all carbon atoms in the α-PEG-β-methyl-6-hydroxyhexyl maleamidic acid can find respective assignments in ¹³C NMR. Among them, the signal peak near 71.2 ppm was assigned to carbon atoms in the backbone of polyethylene glycol, the peak at 173.9 ppm was a signal peak of a carbon atom in an ester bond generated in an acyl chlorination reaction, the signal at 7.9 ppm was assigned to a carbon atom of a methyl group in an acid anhydride substituent, and the newly occurring signal at 20.0-40.0 ppm was assigned to a part of carbon atoms of methylene groups in 6-amino-1 -hexanol. The results of ¹³C NMR further demonstrated the correctness of the structure of the α-PEG-β-methyl-6-hydroxyhexyl maleamidic acid produced.

¹H NMR spectra of α-PEG-β-methyl-6-hydroxyhexyl maleamidic acids having different molecular weights can be seen from FIG. 12. Their signal peaks of protons were all similar to those in FIG. 10 (A), demonstrating that this method is suitable for synthesizing all α-PEG-β-methyl-6-hydroxyhexyl maleamidic acids having different molecular weights.

### Example 4

### Synthesis of α-PEG-6-Hydroxyhexyl Maleamidic Acid

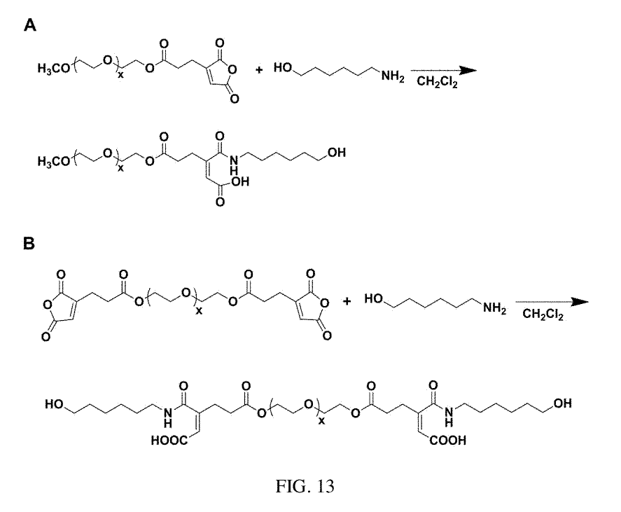

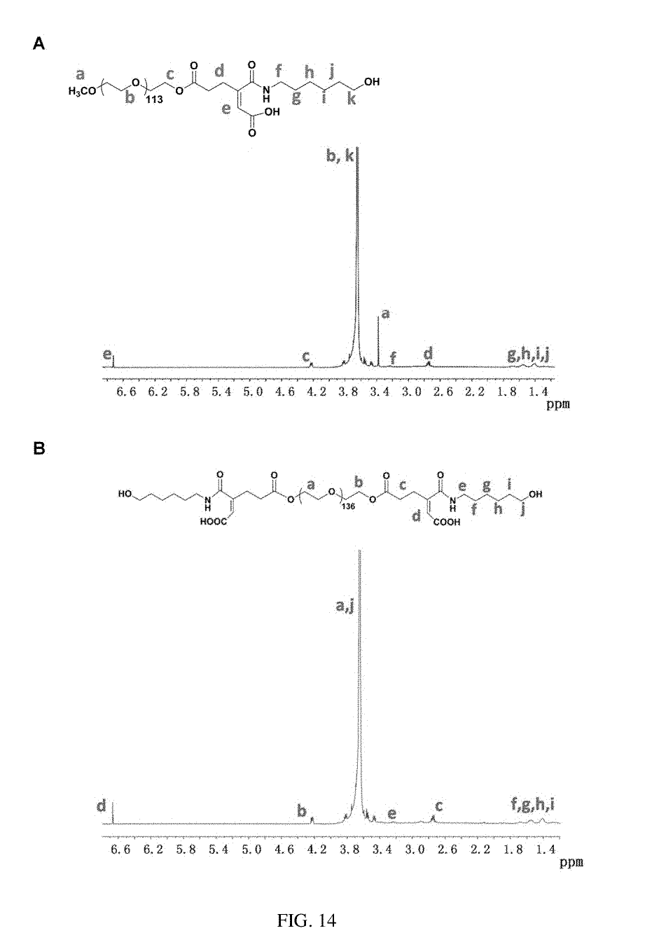

The chemical structure and synthetic route of α-PEG-6-hydroxyhexyl maleamidic acid were as shown in FIG. 13. The resultant product was represented by mPEG-Dlink-OH or HO-Dlink-PEG-Dlink-OH.

A PEG derivative terminated with maleic anhydride and 6-amino-1-hexanol were completely dissolved in anhydrous CH₂Cl₂ together at 25° C., and reaction was performed under stirring (the concentration of the polymer was 0.1 M, the molar ratio of hydroxy groups of 6-amino-1 -hexanol to polyethylene glycol was 3:1). After reaction for 12 h, a saturated NaCl solution was continuously added to extract twice. The organic phase was collected, and precipitated with excessive anhydrous ethyl ether at 0° C. After suction filtration under reduced pressure, a solid was dried under vacuum to a constant weight.

The α-PEG-6-hydroxyhexyl maleamidic acid synthesized as described above was subjected to ¹H NMR analysis to measure its molecular structure, and the ¹H NMR spectra can be seen in FIG. 14. The α-PEG-6-hydroxyhexyl maleamidic acid described above was subjected to ¹³C NMR analysis to further confirm its molecular structure, and ¹³C NMR can be seen in FIG. 15. α-PEG-β-methyl-6-hydroxyhexyl maleamidic acids having different molecular weights were characterized by hydrogen nuclear magnetic resonance spectra, and the results can be seen in FIG. 16.

In FIG. 14 (A), all the signal peaks in hydrogen nuclear magnetic resonance spectra were marked by letters a-k, which were sequentially assigned to respective protons of the corresponding product. Due to the introduction of 6-amino-1-hexanol, new proton signals were found at 3.24 ppm and 1.20-1.80 ppm and can be correctly assigned to protons of reacted 6-amino-1-hexanol. The reaction efficiency of 6-amino-1 -hexanol and an acid anhydride group was obtained by calculating the integrated areas through the signal peak of a methyl group of polyethylene glycol monomethyl ether at 3.37 ppm and the multiple peak at 1.20-1.80 ppm, demonstrating the efficiency of the ring opening reaction was more than 96%.

In FIG. 14 (B), since a polyethylene glycol having two hydroxy groups was used, protonic hydrogen of the terminal methyl group in polyethylene glycol monomethyl ether was absent, while other signals of protons were consistent with those in FIG. 14 (A), and it was indicated by the calculation of integrated areas that the ring opening efficiency of maleic anhydride was higher than 96%.

In FIG. 15, all carbon atoms in the α-PEG-6-hydroxyhexyl maleamidic acid can find respective assignments in ¹³C NMR. Among them, the signal peak near 71.1 ppm was attributed to carbon atoms in the backbone of polyethylene glycol, the peak at 173.9 ppm was a signal peak of a carbon atom in an ester bond generated in an acyl chlorination reaction, and the signal at 20.0-40.0 ppm may be assigned to a part of carbon atoms of methylene groups in 6-amino-l-hexanol. Compared to FIG. 11, the signal at 7.9 ppm assigned to a carbon atom of a methyl group in an acid anhydride substituent disappeared. The results of ¹³C NMR further demonstrated the correctness of the structure of the PEG derivative terminated with maleic anhydride produced.

¹H NMR spectra of α-PEG-6-hydroxyhexyl maleamidic acids having different molecular weights can be seen from FIG. 16. Their signal peaks of protons were all similar to those in FIG. 14 (A), demonstrating that this method is suitable for synthesizing all α-PEG-6-hydroxyhexyl maleamidic acids having different molecular weights.

### Example 5

### Synthesis of Polyethylene Glycol-Dlinkₘ-Polylactide Copolymer Bridged by Acid-Catalytically Hydrolyzable Amide Bond Dlinkₘ

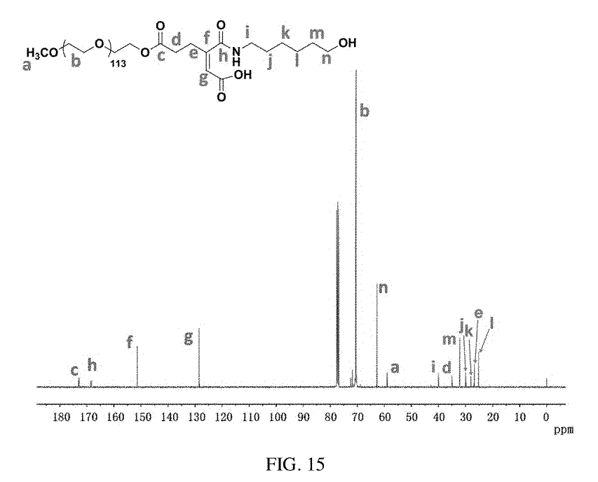

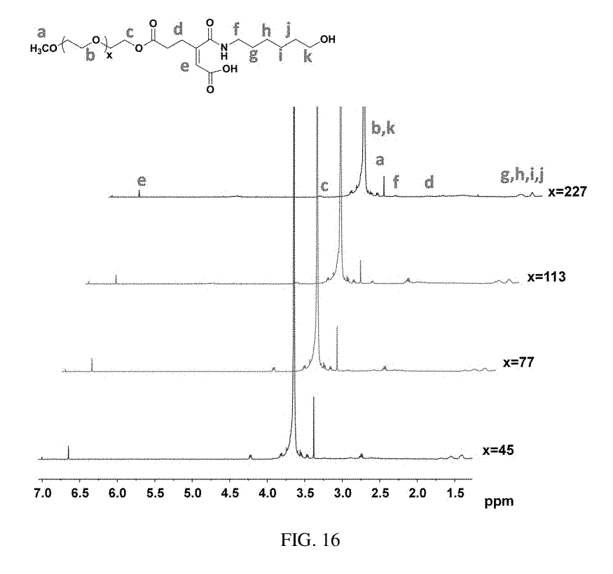

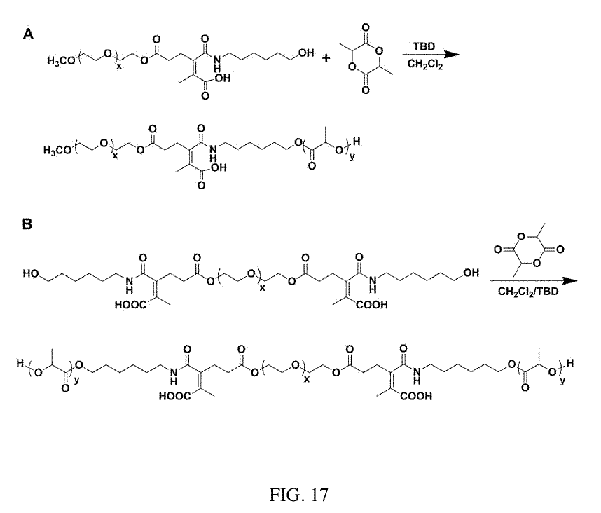

The chemical structure and synthetic route of a polyethylene glycol-Dlinkₘ-polylactide copolymer bridged by an acid-catalytically hydrolyzable amide bond Dlinkₘ were as shown in FIG. 17.

Dlinkₘ bridged polyethylene glycol-Dlinkₘ-polylactide block polymers having different molecular weights were formed by initiating the polymerization of D,L-LA monomers using α-PEG-β-methyl-6-hydroxyhexyl maleamidic acid as an initiator under a solution condition. D,L-LA and macromolecular initiators were dried under vacuum overnight before use. Polyethylene glycol-Dlinkₘ-polylactide block polymers having different molecular weights may be obtained by adjusting the feeding ratio of the monomers to the macromolecular initiator in the process of reaction. 1,5,7-triazabicyclo[4.4.01]dec-5-ene (TBD) belonged to organic heterocyclic non-metal catalysts and has a relatively high catalytic efficiency. It was demonstrated to be relatively suitable for ring opening polymerization of cyclic monomers such as lactones, cyclic diesters, and the like. The specific experimental steps for synthesis were as follows.

Polymerization reaction was performed in an inert gas glovebox (purchased from M. Braun Inertgas Systems (Shanghai) Co., Ltd.), wherein the concentrations of O₂ and H₂O are both less than 0.1 ppm, and the specific experimental steps for synthesis were as follows by exemplifying mPEG-Dlinkₘ-OH or HO-Dlinkₘ-PEG-Dlinkₘ-OH used as an initiator.

1) A round-bottomed flask, in which reaction was performed, was subjected to several treatments of evacuation, flame-drying, and charging with nitrogen gas and placed in a glovebox.

2) Feeding was performed according to the proportions in Table 1: mPEG-Dlinkₘ-OH (or HO-Dlinkₘ-PEG-Dlinkₘ-OH), a D,L-LA monomer, CH₂Cl₂, and TBD were added to the flask, and reaction was performed under stirring at 0° C.

3) After completion of the reaction, the system was concentrated with a rotary evaporator and precipitated twice with a mixed solvent of ethyl ether and methanol (ethyl ether:methanol=20:1, v/v, 100 mL) at 0° C., and the precipitate was collected and dried by suction with an oil pump to a constant weight, so as to obtain a product.

TABLE 1
Synthesis of Dlinkₘ bridged polyethylene glycol-Dlinkₘ-polylactide
copolymers with different feeding ratios (mass ratios)

| | Initiator | CH₂Cl₂ | D,L-LA | TBD |
|---|---|---|---|---|
| Initiator | (g) | (mL) | (g) | (mg) |
| mPEG₁₁₃-Dlinkₘ-OH | 0.52 | 5.0 | 0.41 | 14.6 |
| mPEG₁₁₃-Dlinkₘ-OH | 0.51 | 5.0 | 0.65 | 21.9 |
| mPEG₁₁₃-Dlinkₘ-OH | 0.52 | 10.0 | 1.14 | 30.8 |
| mPEG₁₁₃-Dlinkₘ-OH | 0.50 | 15.0 | 2.36 | 34.1 |
| mPEG₄₅-Dlinkₘ-OH | 0.21 | 5.0 | 0.29 | 7.9 |
| mPEG₄₅-Dlinkₘ-OH | 0.22 | 15.0 | 2.03 | 13.4 |
| mPEG₇₇-Dlinkₘ-OH | 0.34 | 5.0 | 0.39 | 14.5 |
| mPEG₇₇-Dlinkₘ-OH | 0.34 | 15.0 | 2.15 | 29.3 |
| mPEG₂₂₅-Dlinkₘ-OH | 1.02 | 20.0 | 0.31 | 16.4 |
| mPEG₂₂₅-Dlinkₘ-OH | 1.01 | 25.0 | 2.29 | 37.6 |
| mPEG₄₅₀-Dlinkₘ-OH | 2.03 | 20.0 | 0.28 | 11.4 |
| mPEG₄₅₀-Dlinkₘ-OH | 2.00 | 25.0 | 2.35 | 34.7 |
| HO-Dlinkₘ-PEG₁₃₆-Dlinkₘ-OH | 0.61 | 10.0 | 1.34 | 18.7 |

Number-average molecular weights and polydispersity index (PDIs) of polyethylene glycol-polylactide block polymers were analyzed by using a gel permeation chromatography (GPC) method with polystyrene as a standard. GPC spectra can be seen in FIG. 18, and number-average molecular weights and molecular weight distribution PDIs can be seen in Table 2.

As can be seen from FIG. 18, GPC spectra of block polymers were all single peaks. Furthermore, there was no phenomenon of tailing, that is, there was no signal peak of a macromolecular initiator. It was demonstrated that macromolecular initiators had been completely depleted and expected block copolymers were obtained.

TABLE 2
Molecular weights and compositions of Dlinkₘ bridged
polyethylene glycol-polylactide copolymers

Block copolymer

| Block copolymer | Mₙ^{a} | Mₙ^{b} | PDI^{c} |
|---|---|---|---|
| mPEG₁₁₃-Dlinkₘ-PDLLA₄₂ | 5000-3020 | 9600 | 1.10 |
| mPEG₁₁₃-Dlinkₘ-PDLLA₇₁ | 5000-5100 | 13000 | 1.12 |
| mPEG₁₁₃-Dlinkₘ-PDLLA₁₄₂ | 5000-10220 | 18900 | 1.21 |
| mPEG₁₁₃-Dlinkₘ-PDLLA₂₈₉ | 5000-20800 | 24900 | 1.25 |
| mPEG₄₅-Dlinkₘ-PDLLA₃₁ | 2000-2230 | 5700 | 1.12 |
| mPEG₄₅-Dlinkₘ-PDLLA₂₈₁ | 2000-20230 | 26300 | 1.23 |
| mPEG₇₇-Dlinkₘ-PDLLA₂₉ | 3400-2080 | 6400 | 1.17 |
| mPEG₇₇-Dlinkₘ-PDLLA₂₇₅ | 3400-19800 | 28140 | 1.21 |
| mPEG₂₂₅-Dlinkₘ-PDLLA₃₀ | 10000-2160 | 15480 | 1.18 |
| mPEG₂₂₅-Dlinkₘ-PDLLA₂₉₃ | 10000-21090 | 34620 | 1.26 |
| mPEG₄₅₀-Dlinkₘ-PDLLA₃₃ | 20000-2370 | 27300 | 1.27 |
| mPEG₄₅₀-Dlinkₘ-PDLLA₂₆₄ | 20000-19000 | 56480 | 1.19 |
| PDLLA₇₁-Dlinkₘ-PEG₁₃₆- | 5100-6000- | 19400 | 1.18 |
| Dlinkₘ-PDLLA₇₁ | 5100 | | |

^{a}obtained by ¹H NMR;
^{b}obtained by GPC; and
^{c}obtained by GPC.

The Dlinkₘ bridged polyethylene glycol-polylactide copolymers described above were subjected to ¹H NMR analysis to measure their polymerization degrees and number-average molecular weights, and the ¹H NMR spectra can be seen in FIG. 19. The Dlinkₘ bridged polyethylene glycol-polylactide copolymers described above were subjected to ¹³C NMR analysis to further confirm its structure, and the ¹³C NMR spectra can be seen in FIG. 20.

In FIG. 19, protonic hydrogens assigned to a two-block polymer were marked by letters a to g in the ¹H NMR spectra of mPEG-Dlink-PDLLA. The polymerization degree of polylactide was obtained by calculating the integrated area ratio of the multiple peak at 1.58 ppm (assigned to —CH₃ of polylactide) to the single peak at 3.67 ppm (assigned to —OCH₂CH₂— of polyethylene glycol).

FIG. 20 was a ¹³C NMR spectrum of mPEG₁₁₃-Dlinkₘ-PDLLA₇₁, and carbon atoms assigned to a block polymer were marked by letters a to r. Among them, the signal peak at 71.2 ppm was assigned to carbon atoms in the backbone of polyethylene glycol, the signal peak at 168.9 ppm was assigned to a macromolecular initiator and a signal peak of a carbonyl group in the backbone of polylactide, and the signal of a carbon atom of a methyl group in a polylactide block was at 16.7 ppm. The structure of the block polymer was further validated by the results of carbon nuclear magnetic resonance spectra.

### Example 6

### Synthesis of Polyethylene Glycol-Dlink-Polylactide Copolymer Bridged by Acid-Catalytically Hydrolyzable Amide Bond Dlink

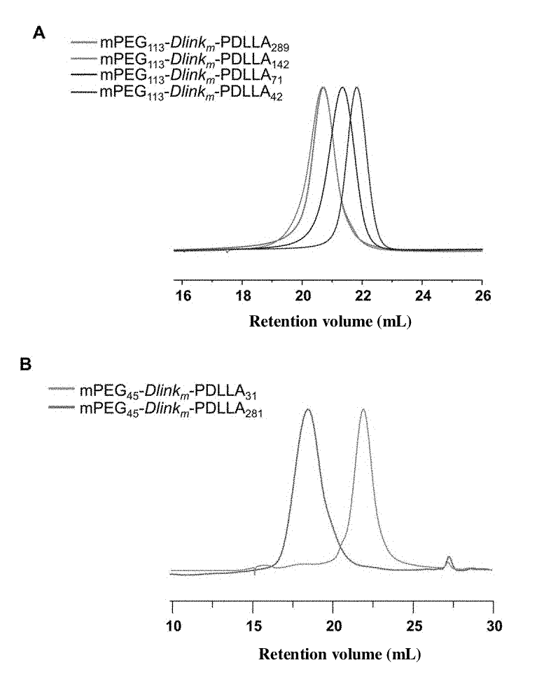

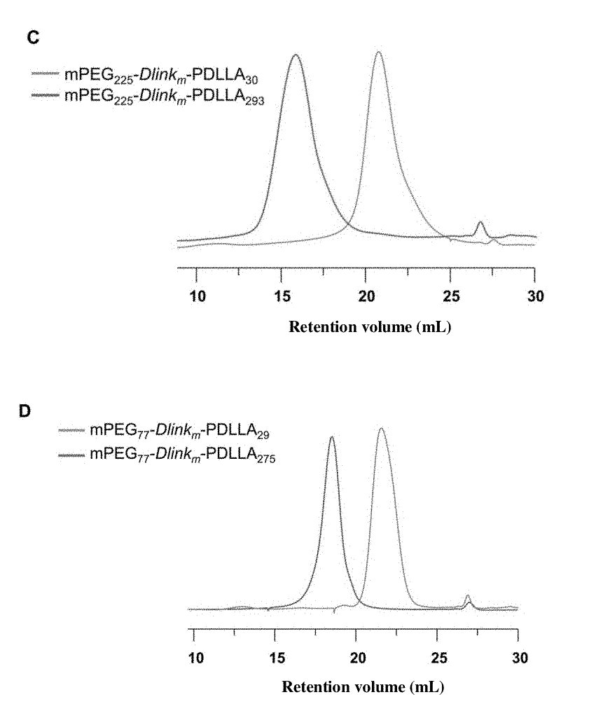

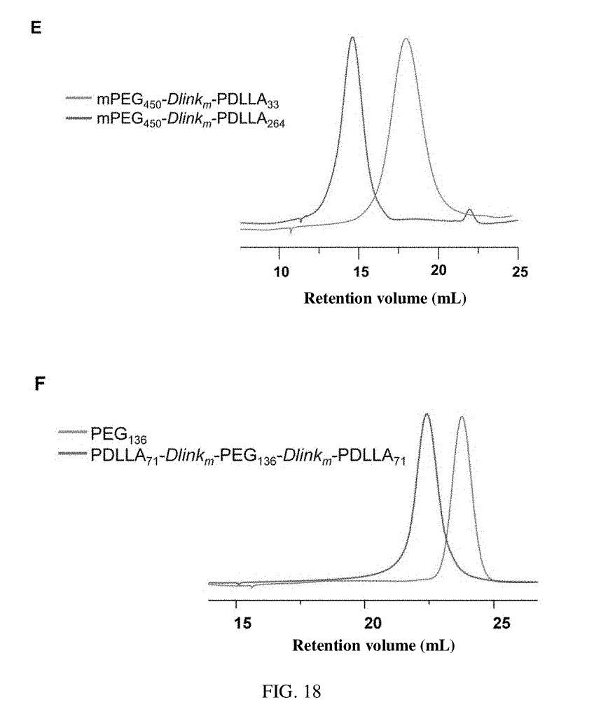

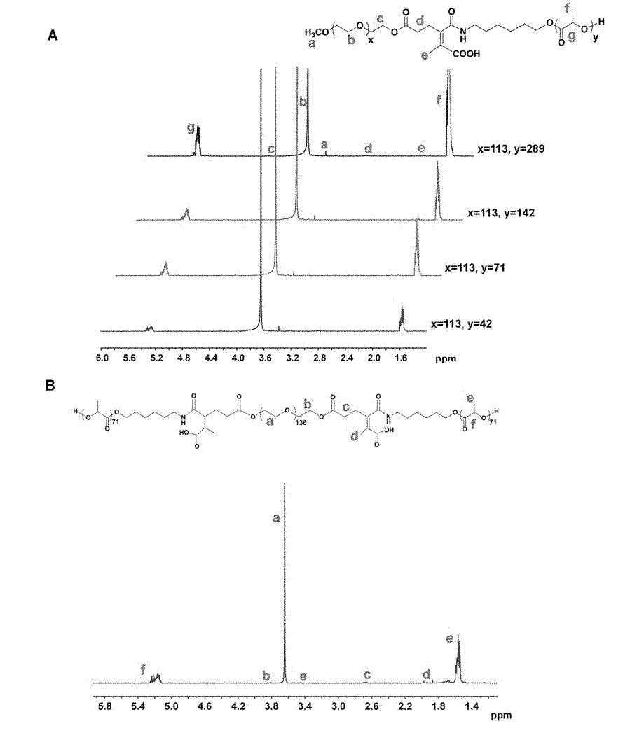

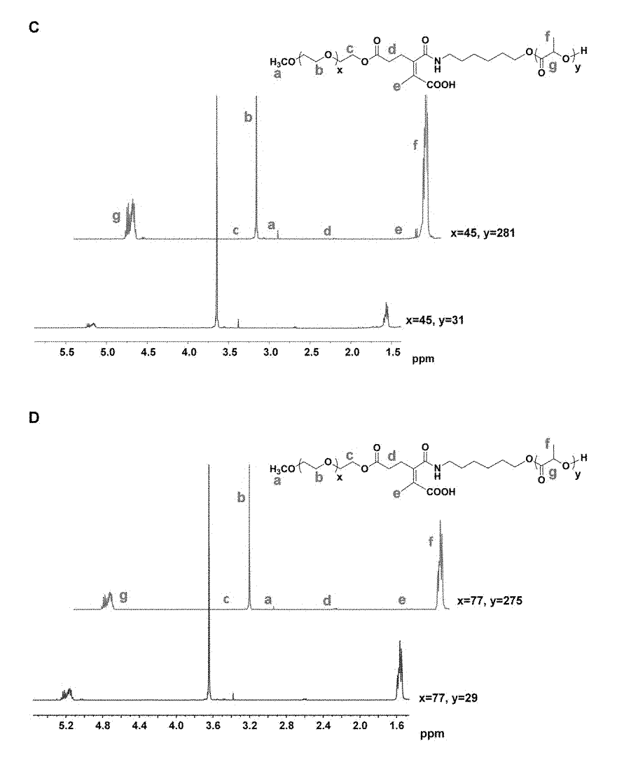

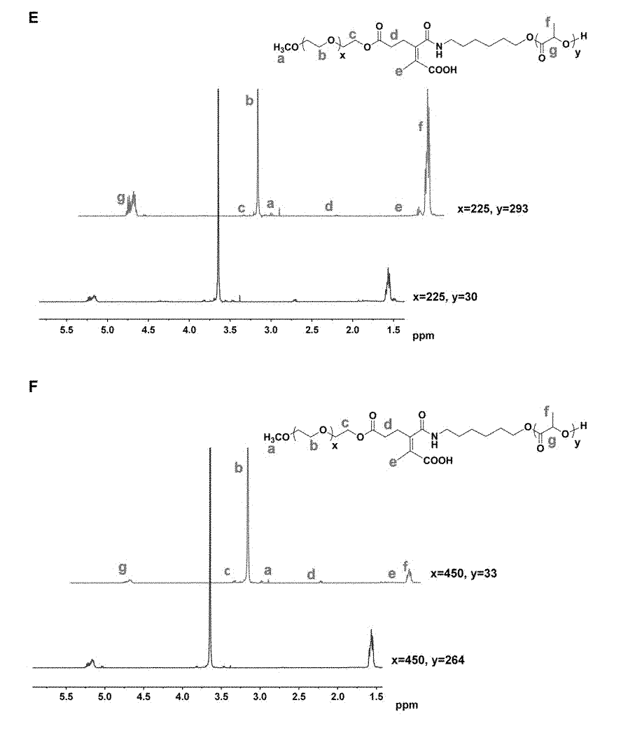

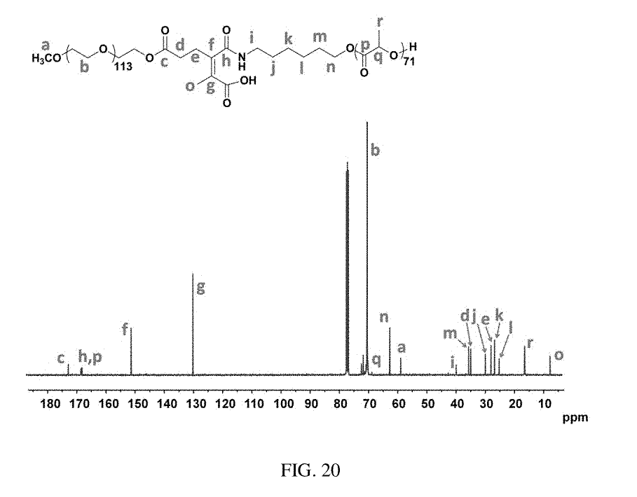

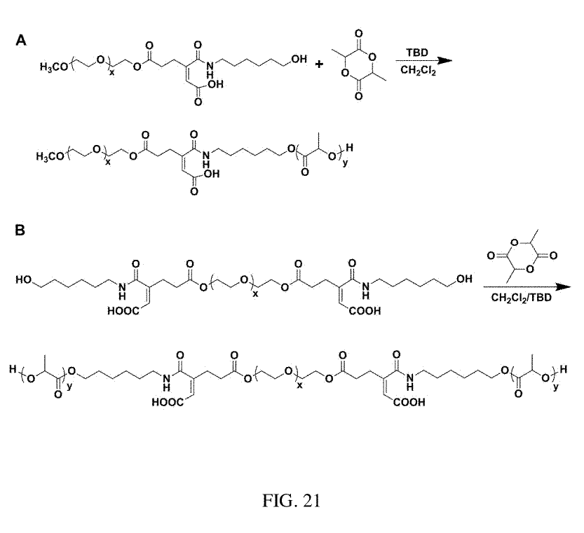

The chemical structure and synthetic route of a polyethylene glycol-Dlink-polylactide copolymer bridged by an acid-catalytically hydrolyzable amide bond Dlink were as shown in FIG. 21.

Dlink bridged polyethylene glycol-Dlink-polylactide block polymers having various molecular weights were formed by initiating the polymerization of D,L-LA monomers using α-PEG-6-hydroxyhexyl maleamidic acid as an initiator under a solution condition. D,L-LA and macromolecular initiators were dried under vacuum overnight before use. Polyethylene glycol-Dlink-polylactide block polymers having different molecular weights may be obtained by adjusting the feeding ratio of the monomers to the macromolecular initiator in the process of reaction. The specific experimental steps for synthesis were as follows.

Polymerization reaction was performed in an inert gas glovebox, wherein the concentrations of O₂ and H₂O are both less than 0.1 ppm, and the specific experimental steps for synthesis were as follows by exemplifying mPEG₁₁₃-Dlink-OH or HO-Dlink-PEG₁₃₆-Dlink-OH used as an initiator.

1) A round-bottomed flask, in which reaction was performed, was subjected to several treatments of evacuation, flame-drying, and charging with nitrogen gas and placed in a glovebox.

2) Feeding was performed according to the proportions in Table 3: mPEG₁₁₃-Dlink-OH (or HO-Dlink-PEG136-Dlink-OH), a D,L-LA monomer, CH₂Cl₂, and TBD were added to the flask, and reaction was performed under stirring at 0° C.

3) After completion of the reaction, the system was concentrated with a rotary evaporator and precipitated twice with a mixed solvent of ethyl ether and methanol (ethyl ether:methanol=20:1, v/v, 100 mL) at 0° C., and the precipitate was collected and dried by suction with an oil pump to a constant weight, so as to obtain a product.

TABLE 3
Synthesis of Dlink bridged polyethylene glycol-Dlink-polylactide
copolymers with different feeding ratios (mass ratios)

| | Initiator | CH₂Cl₂ | D,L-LA | TBD |
|---|---|---|---|---|
| Initiator | (g) | (mL) | (g) | (mg) |
| mPEG₁₁₃-Dlink-OH | 0.52 | 5.0 | 0.41 | 14.6 |
| mPEG₁₁₃-Dlink-OH | 0.52 | 5.0 | 2.65 | 31.9 |
| HO-Dlink-PEG₁₃₆-Dlink-OH | 0.61 | 10.0 | 1.14 | 21.8 |

Number-average molecular weights and polydispersity index of polyethylene glycol-Dlink-polylactide block polymers were analyzed by using a gel permeation chromatography method with polystyrene as a standard. GPC spectra can be seen in FIG. 22, and number-average molecular weights and molecular weight distribution PDIs can be seen in Table 4.

As can be seen from FIG. 22, GPC spectra of block polymers were all single peaks. Furthermore, there was no phenomenon of tailing, that is, there was no signal peak of a macromolecular initiator. It was demonstrated that macromolecular initiators had been completely depleted and expected block copolymers were obtained.

TABLE 4
Molecular weights and compositions of Dlink bridged
polyethylene glycol-Dlink-polylactide copolymers

| | Block copolymer | | |
|---|---|---|---|
| Block copolymer | Mₙ^{a} | Mₙ^{b} | PDI^{c} |
| mPEG₁₁₃-Dlink-PDLLA₄₂ | 5000-3020 | 11400 | 1.12 |
| mPEG₁₁₃-Dlink-PDLLA₂₈₉ | 5000-20800 | 24900 | 1.18 |
| PDLLA₆₄-Dlink-PEG₁₃₆-Dlink- | 4600-6000- | 17300 | 1.16 |
| PDLLA₆₄ | 4600 | | |

^{a}obtained by ¹H NMR;
^{b}obtained by GPC; and
^{c}obtained by GPC.

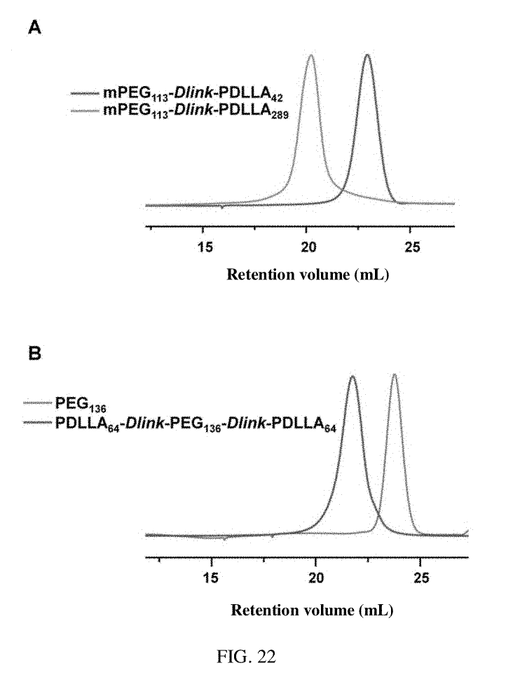

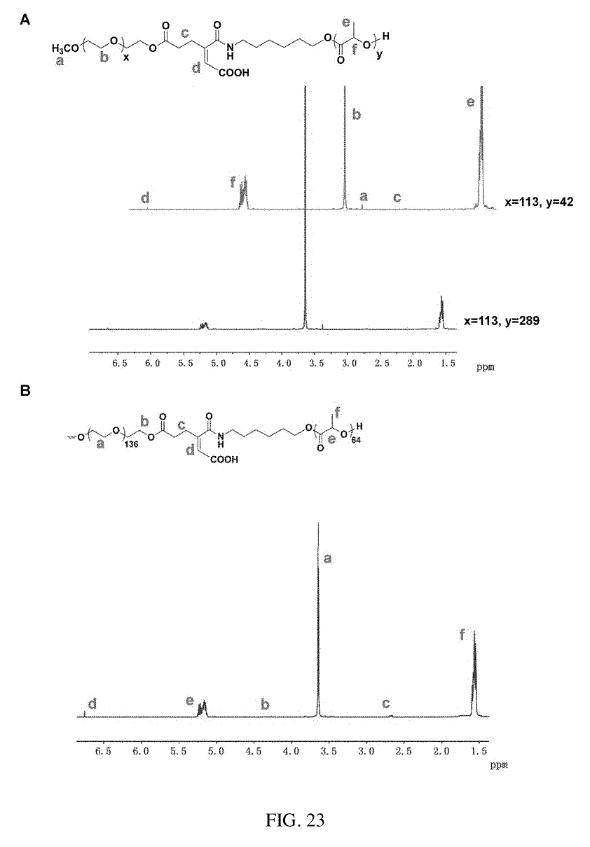

The Dlink bridged polyethylene glycol-Dlink-polylactide copolymers described above were subjected to hydrogen nuclear magnetic resonance spectrum analysis to measure their polymerization degrees and number-average molecular weights, and the ¹H NMR spectra can be seen in FIG. 23. The Dlink bridged polyethylene glycol-Dlink-polylactide copolymers described above were subjected to carbon nuclear magnetic resonance spectrum analysis to further confirm its structure, and the ¹³C NMR spectra can be seen in FIG. 24.

In FIG. 23, protonic hydrogens assigned to a two-block polymer were marked by letters a to f in the ¹H NMR spectra of mPEG-Dlink-PDLLA. The polymerization degree of polylactide was obtained by calculating the integrated area ratio of the multiple peak at 1.58 ppm (assigned to —CH₃ of polylactide) to the single peak at 3.65 ppm (assigned to —OCH₂CH₂— of polyethylene glycol).

FIG. 24 was a ¹³C NMR spectrum of mPEG₁₁₃-Dlink-PDLLA₄₂, and carbon atoms assigned to a two-block polymer were marked by letters a to q. Compared to FIG. 20, the signal peak of carbon of a methyl group in an acid anhydride substituent at 7.8 ppm disappeared, while all other signal peaks were relatively similar. The structure of the block polymer was further validated.

### Example 7

### Synthesis of Dlink (or Dlinkₘ) Bridged Polyethylene Glycol-Dlink-Polycaprolactone (or Polyethylene Glycol-Dlinkₘ-Polycaprolactone) Copolymer

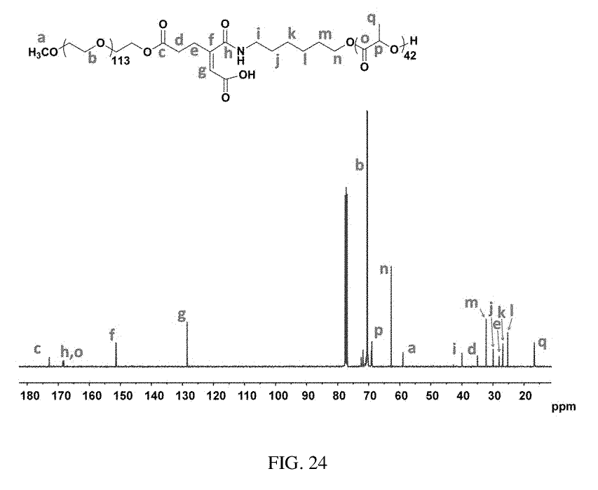

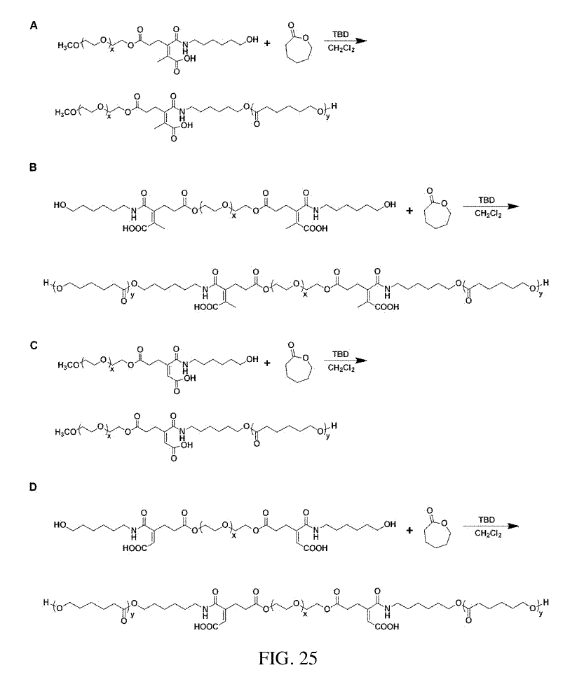

The chemical structure and synthetic route of a Dlink (or Dlinkₘ) bridged polyethylene glycol-Dlink-polycaprolactone (or polyethylene glycol-Dlinkₘ-polycaprolactone) copolymer were as shown in FIG. 25.

Block polymers of polyethylene glycol and polycaprolactone bridged by an acid-sensitive chemical bond having various molecular weights were formed by initiating the polymerization of ε-CL monomers using α-PEG-β-methyl-6-hydroxyhexyl maleamidic acid or α-PEG-6-hydroxyhexyl maleamidic acids as an initiator under a solution condition. Macromolecular initiators were dried under vacuum overnight before use. By adjusting the feeding ratio of ε-CL to the initiator, block polymers of polyethylene glycol and polycaprolactone bridged by an acid-sensitive chemical bond having different molecular weights may be obtained.

Polymerization reaction was performed in an inert gas glovebox, wherein the concentrations of O₂ and H₂O are both less than 0.1 ppm, and the specific experimental steps for synthesis were as follows.

1) A round-bottomed flask, in which reaction was performed, was subjected to several treatments of evacuation, flame-drying, and charging with nitrogen gas and placed in a glovebox.

2) Feeding was performed according to the proportions in Table 5: a macromolecular initiator, an ε-CL monomer, CH₂Cl₂, and TBD were added to the flask, and reaction was performed under stirring at 0° C.

3) After completion of the reaction, the system was concentrated with a rotary evaporator and precipitated twice with a mixed solvent of ethyl ether and methanol (ethyl ether:methanol=20:1, v/v, 100 mL) at 0° C., and the precipitate was collected and dried by suction with an oil pump to a constant weight, so as to obtain a product.

TABLE 5
Synthesis of Dlink (or Dlinkₘ) bridged polyethylene glycol-Dlink-
polycaprolactone (or polyethylene glycol-Dlinkₘ-polycaprolactone)
copolymers with different feeding ratios (mass ratios)

| | Initiator | CH₂Cl₂ | ε-CL | TBD |
|---|---|---|---|---|
| Initiator | (g) | (mL) | (g) | (mg) |
| mPEG₇₇-Dlinkₘ-OH | 0.34 | 5.0 | 0.36 | 18.1 |
| mPEG₇₇-Dlinkₘ-OH | 0.36 | 5.0 | 0.70 | 24.2 |
| mPEG₇₇-Dlinkₘ-OH | 0.35 | 10.0 | 1.20 | 34.7 |
| mPEG₇₇-Dlink-OH | 0.34 | 5.0 | 0.34 | 15.7 |
| mPEG₇₇-Dlink-OH | 0.36 | 15.0 | 2.44 | 46.9 |
| HO-Dlinkₘ-PEG₁₃₆-Dlinkₘ-OH | 0.62 | 10.0 | 1.38 | 28.9 |
| HO-Dlink-PEG₁₃₆-Dlink-OH | 0.61 | 10.0 | 1.47 | 26.4 |

Number-average molecular weights and molecular weight distributions of the copolymer were analyzed by using a gel permeation chromatography method with polystyrene as a standard. GPC spectra can be seen in FIGS. 26 and 27, and number-average molecular weights and molecular weight distribution PDIs can be seen in Table 6.

As can be seen, from FIGS. 26 and 27, there was no phenomenon of tailing caused by the presence of macromolecular initiators. It was demonstrated that macromolecular initiators had been completely depleted and expected two-block copolymers were obtained.

TABLE 6
Molecular weights and compositions of Dlink (or
Dlinkₘ) bridged polyethylene glycol-Dlink-polycaprolactone (or
polyethylene glycol-Dlinkₘ-polycaprolactone) copolymers

| | Block copolymer | | |
|---|---|---|---|
| Block copolymer | Mₙ^{a} | Mₙ^{b} | PDI^{c} |
| mPEG₇₇-Dlinkₘ-PCL₁₈ | 3400-2050 | 7900 | 1.08 |
| rnPEG₇₇-Dlinkₘ-PCL₄₆ | 3400-5240 | 10300 | 1.12 |
| mPEG₇₇-Dlinkₘ-PCL₉₅ | 3400-10830 | 14500 | 1.11 |
| mPEG₇₇-Dlink-PCL₂₄ | 3400-2740 | 8400 | 1.11 |
| mPEG₇₇-Dlink-PCL₁₇₉ | 3400-20406 | 29200 | 1.24 |
| PCL₃₅-Dlinkₘ-PEG₁₃₆-Dlinkₘ-PCL₃₅ | 3990-6000-3990 | 10300 | 1.17 |
| PCL₃₂-Dlink-PEG₁₃₆-Dlink-PCL₃₂ | 3640-6000-3640 | 14900 | 1.15 |

^{a}obtained by ¹H NMR;
^{b}obtained by GPC; and
^{c}obtained by GPC.

The Dlinkₘ and Dlink bridged copolymers described above were subjected to hydrogen nuclear magnetic resonance spectrum analysis to measure their polymerization degrees and number-average molecular weights, and the ¹H NMR spectra can be seen in FIGS. 28 and 29. The Dlinkₘ and Dlink bridged copolymers described above were subjected to carbon nuclear magnetic resonance spectrum analysis, and the ¹³C NMR spectra can be seen in FIGS. 30 and 31.

In FIGS. 28 and 29, protonic hydrogens assigned to a block polymer were marked by letters a to m. The polymerization degree of polycaprolactone was obtained by calculating the integrated area ratio of the multiple peak at 4.08 ppm (assigned to —OC(O)CH2 of polycaprolactone) to the single peak at 3.65 ppm (assigned to —OCH₂CH₂— of polyethylene glycol).

FIG. 30 was a ¹³C NMR spectrum of mPEG₇₇-Dlinkₘ-PCL₉₅, and carbon atoms assigned to a block polymer were marked by letters a to u. Among them, the signal peak at 72.1 ppm was assigned to carbon atoms in the backbone of polyethylene glycol, the signal peaks at 174.2 ppm and 169.1 ppm were attributed to a macromolecular initiator and a signal peak of a carbonyl group in the backboneof polycaprolactone, the signal peaks at 20.0-40.0 ppm were assigned to signals of carbon atoms of a methylene group in a polycaprolactone block and a methylene group in 6-amino-hexanol, and the signal peak of a carbon atom of a methyl group in Dlinkₘ was at 9.9 ppm. The structure of the block polymer was further validated by the results of carbon nuclear magnetic resonance spectra.

FIG. 31 was a ¹³C NMR spectrum of mPEG77-Dlink-PCL70, and carbon atoms assigned to a block polymer were marked by letters a to t. Compared to FIG. 30, the signal peak assigned to a methyl group in Dlinkₘ near 7.84 ppm disappeared, while other signal peaks were similar.

### Example 8

### Synthesis of Dlinkₘ (or Dlink) Bridged Polyethylene Glycol-Dlinkₘ-Poly(Lactic-co-Glycolic Acid) (or Polyethylene Glycol-Dlink-Poly(Lactic-co-Glycolic Acid)) Copolymer

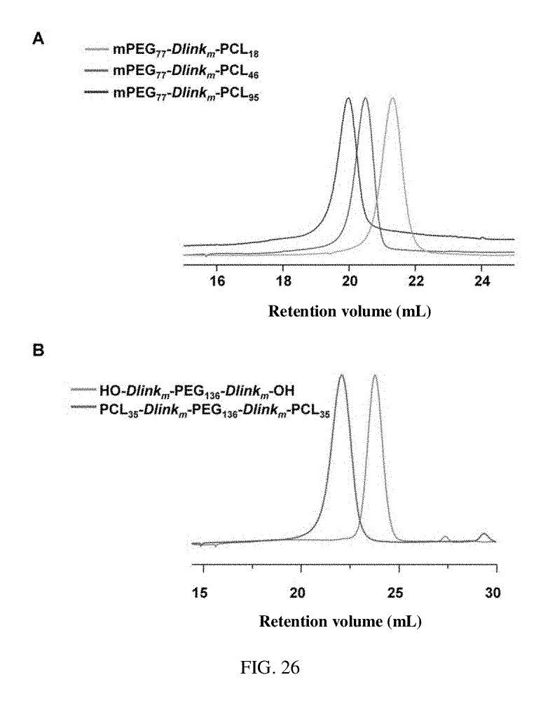

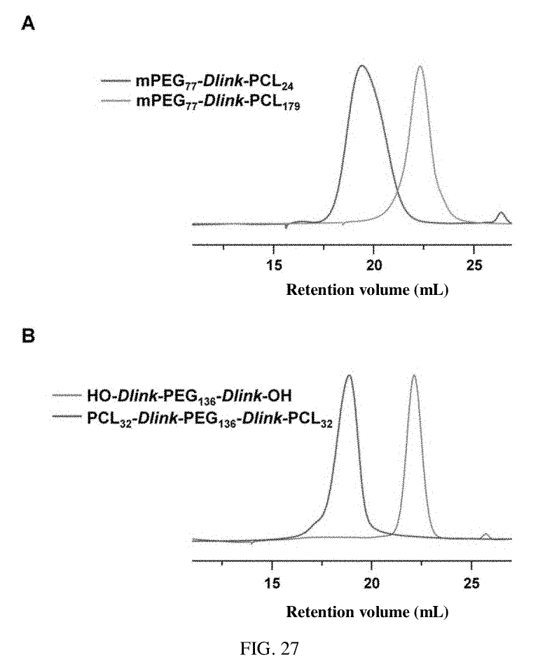

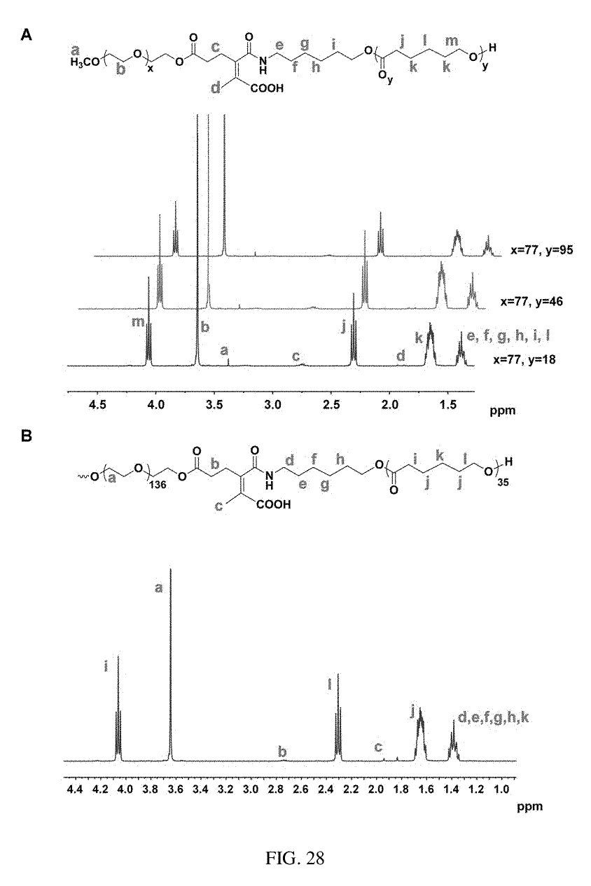

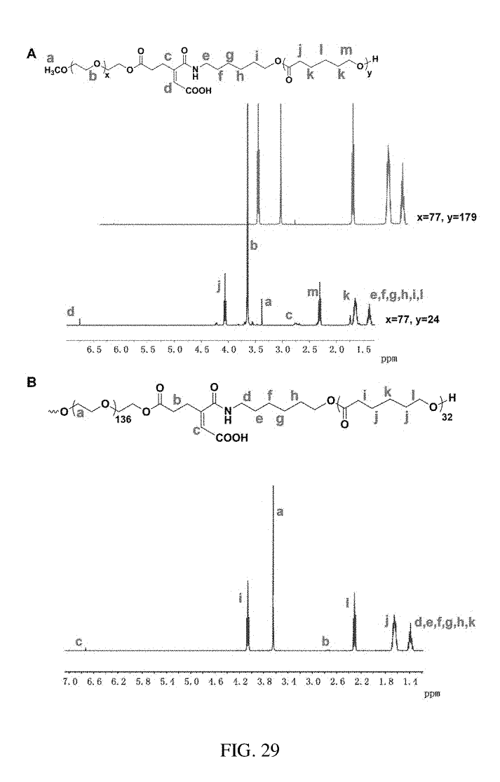

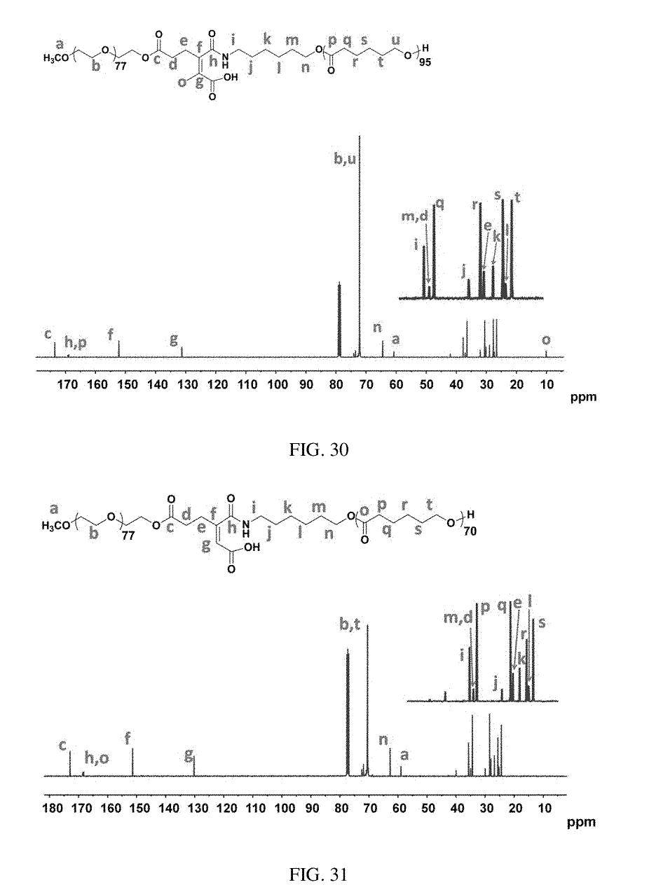

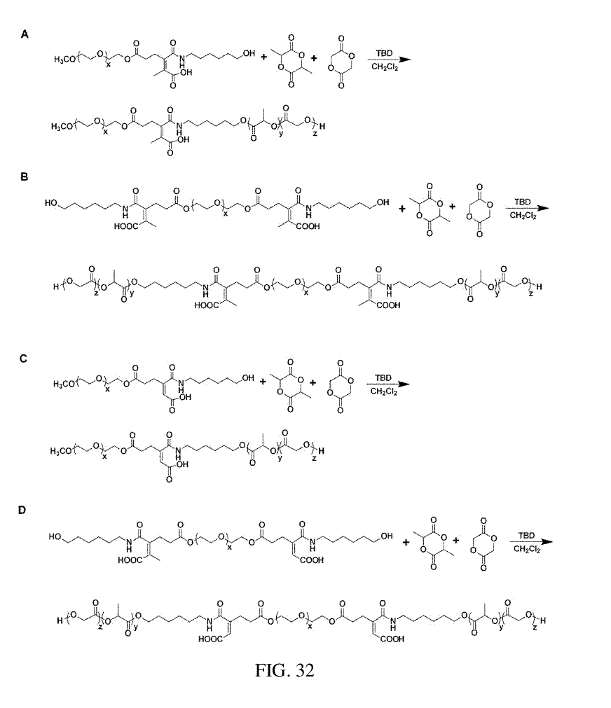

The chemical structure and synthetic route of a Dlinkₘ (or Dlink) bridged polyethylene glycol-Dlinkₘ-poly(lactic-co-glycolic acid) (or polyethylene glycol-Dlink-poly(lactic-co-glycolic acid)) copolymer were as shown in FIG. 32.

Block polymers of polyethylene glycol and poly(lactic-co-glycolic acid) bridged by an acid-sensitive chemical bond having various molecular weights were formed by initiating the polymerization of mixed monomers of D,L-LA and GA using α-PEG-β-methyl-6-hydroxyhexyl maleamidic acid or α-PEG-6-hydroxyhexyl maleamidic acids as an initiator under a solution condition, wherein the ratio of repeating units of polylactide to polyglycolic acid in the product of interest was 3:1. D,L-LA and GA monomers and macromolecular initiators were dried under vacuum overnight before use. By adjusting the feeding ratio of monomers to the initiator, block polymers of polyethylene glycol and poly(lactic-co-glycolic acid) bridged by an acid-sensitive chemical bond having different molecular weights may be obtained.

Polymerization reaction was performed in an inert gas glovebox, wherein the concentrations of O₂ and H₂O are both less than 0.1 ppm, and the specific experimental steps for synthesis were as follows.

1) A round-bottomed flask, in which reaction was performed, was subjected to several treatments of evacuation, flame-drying, and charging with nitrogen gas and placed in a glovebox.

2) Feeding was performed according to the proportions in Table 7: a macromolecular initiator, D,L-LA and GA monomers, CH₂Cl₂, and TBD were added to the flask, and reaction was performed under stirring at 0° C.

3) After completion of the reaction, the system was concentrated with a rotary evaporator and precipitated twice with a mixed solvent of ethyl ether and methanol (ethyl ether:methanol=20:1, v/v, 100 mL) at 0° C., and the precipitate was collected and dried by suction with an oil pump to a constant weight, so as to obtain a product.

TABLE 7
Synthesis of Dlinkₘ (or Dlink) bridged polyethylene glycol-
Dlinkₘ-poly(lactic-co-glycolic acid) (or polyethylene
glycol-Dlink-poly(lactic-co-glycolic acid)) copolymers
with different feeding ratios (mass ratios)

| | Initiator | CH₂Cl₂ | D,L-LA/GA | TBD |
|---|---|---|---|---|
| Initiator | (g) | (mL) | (g) | (mg) |
| mPEG₄₅-Dlinkₘ-OH | 0.22 | 5.0 | 0.20/0.08 | 12.8 |
| mPEG₄₅-Dlinkₘ-OH | 0.22 | 5.0 | 0.82/0.30 | 24.6 |
| mPEG₂₂₅-Dlinkₘ-OH | 1.02 | 20.0 | 1.25/0.52 | 32.9 |
| mPEG₄₅₀-Dlinkₘ-OH | 2.01 | 25.0 | 1.67/0.71 | 44.5 |
| mPEG₄₅-Dlink-OH | 0.21 | 10.0 | 0.24/0.11 | 15.7 |
| mPEG₄₅-Dlink-OH | 0.21 | 20.0 | 1.59/0.61 | 26.3 |
| HO-Dlinkₘ-PEG₁₃₆-Dlinkₘ-OH | 0.62 | 15.0 | 0.82/0.29 | 28.9 |
| HO-Dlink-PEG₁₃₆-Dlink-OH | 0.62 | 15.0 | 0.86/0.32 | 24.4 |

Number-average molecular weights and polydispersity index of the copolymer were analyzed by using a gel permeation chromatography method with polystyrene as a standard. GPC spectra can be seen in FIGS. 33 and 34, and number-average molecular weights and molecular weight distribution PDIs can be seen in Table 8.

As can be seen, from FIGS. 33 and 34, GPC spectra of copolymers were single peaks, and there was no phenomenon of tailing caused by the presence of macromolecular initiators. It was demonstrated that macromolecular initiators had been completely depleted and expected two-block copolymers were obtained.

TABLE 8
Molecular weights and compositions of
Dlinkₘ and Dlink bridged block copolymers of
polyethylene glycol and poly(lactic-co-glycolic acid)

| | Block copolymer | | |
|---|---|---|---|
| Block copolymer | Mₙ^{a} | Mₙ^{b} | PDI^{c} |
| mPEG₄₅-Dlinkₘ-PLGA_{22/8} | 2000-2040 | 6720 | 1.09 |
| mPEG₄₅-Dlinkₘ-PLGA_{112/39} | 2000-10300 | 16200 | 1.13 |
| mPEG₂₂₅-Dlinkₘ-PLGA_{164/56} | 10000-15050 | 25700 | 1.15 |
| mPEG₄₅₀-Dlinkₘ-PLGA_{225/74} | 20000-20500 | 38200 | 1.24 |
| mPEG₄₅-Dlink-PLGA_{20/7} | 2000-1860 | 5600 | 1.18 |
| mPEG₄₅-Dlink-PLGA_{201/66} | 2000-18400 | 24100 | 1.22 |
| PLGA_{56/18}-Dlinkₘ-PEG₁₃₆-Dlinkₘ- | 5110-6000- | 19800 | 1.16 |
| PLGA_{56/18} | 5110 | | |
| PLGA_{60/21}-Dlink-PEG₁₃₆-Dlink- | 5600-6000- | 17500 | 1.19 |
| PLGA_{60/21} | 5600 | | |

^{a}obtained by ¹H NMR;
^{b}obtained by GPC; and
^{c}obtained by GPC.

The Dlinkₘ and Dlink bridged block copolymers of polyethylene glycol and poly(lactic-co-glycolic acid) described above were subjected to hydrogen nuclear magnetic resonance spectrum analysis to measure their polymerization degrees and number-average molecular weights, and the ¹H NMR spectra can be seen in FIGS. 35 and 36. The Dlinkₘ and Dlink bridged block copolymers of polyethylene glycol and poly(lactic-co-glycolic acid) described above were subjected to carbon nuclear magnetic resonance spectrum analysis, and the ¹³C NMR spectra can be seen in FIGS. 37 and 38.

In FIGS. 35 and 36, protonic hydrogens assigned to a block polymer were marked by letters a to g. Multiple peaks at 1.59 ppm, 4.83 ppm, and 5.22 ppm were assigned to protons of the poly(lactic-co-glycolic acid) block. The polymerization degree of poly(lactic-co-glycolic acid) was obtained by calculating the integrated area ratio of the multiple peak at 1.59 ppm (assigned to —CH₃ in polylactide), the multiple peak at 4.83 ppm (assigned to —CH₂— in polyglycolic acid), and the single peak at 3.65 ppm (assigned to —OCH₂CH₂— of polyethylene glycol).

FIG. 37 was a ¹³C NMR spectrum of mPEG45-Dlinkₘ-PLGA_{112/39,} and carbon atom signals assigned to a block polymer were marked by letters a to t. Among them, the signal peak at 72.4 ppm was assigned to carbon atoms in the backbone of polyethylene glycol, the signal peaks at 20.0-40.0 ppm were assigned to signals of carbon atoms of methylene groups in a polycaprolactone block and 6-amino-hexanol, the signal peak at 16.7 ppm was assigned to a carbon atom of a methyl group in poly(lactic-co-glycolic acid), and the signal peak of a methyl group in Dlinkₘ was at 7.9 ppm. The structure of the block polymer was further validated by the results of carbon nuclear magnetic resonance spectra.

FIG. 38 was a ¹³C NMR spectrum of mPEG₇₇-Dlink-PLGA_{20/7}, and carbon atoms assigned to a block polymer were marked by letters a to s. Compared to FIG. 37, the signal peak assigned to a methyl group in Dlinkₘ near 7.9 ppm disappeared, while other signal peaks were similar.

### Example 9

### Production of Nanoparticles

Micelle- or vesicle-like nanoparticles may be formed from amphipathic polyethylene glycol-aliphatic polyester in water under certain conditions by various methods. At the meanwhile, a hydrophobic drug molecule or fluorochrome may be entrapped by its hydrophobic core, and the hydrophilic structure may bond to siRNAs with the assistance of a cationic lipid. The following nanoparticles were produced by using different emulsification methods in this Example.

For producing empty nanoparticles, mPEG-Dlinkₘ-PDLLA was taken as an example, and the specific method was as follows. mPEG₁₁₃-Dlinkₘ-PDLLA₁₄₂ having a mass of 10 mg was dissolved in 200 μL of ethyl acetate. 1 mL of water was added to the solution described above, and ultrasonication was then performed in an ice bath for 1 min (130 W, operation for 4 s and stop for 2 s, and 60 s in total). 2 mL of water was further added, and after transferring to a round-bottomed flask, evaporation was immediately performed under reduced pressure to remove ethyl acetate.

For producing drug-carrying nanoparticles, mPEG-Dlink-PDLLA was taken as an example, and the specific method was as follows. mPEG₁₁₃-Dlink-PDLLA₁₄₂ having a mass of 10 mg and 1 mg of docetaxel (DTXL) were dissolved in 200 μL of ethyl acetate. 1 mL of water was added to the oil phase described above, and ultrasonication was then performed in an ice bath for 1 min (130 W, operation for 4 s and stop for 2 s, and 60 s in total). 2 mL of water was further added, and after transferring to a round-bottomed flask, evaporation was immediately performed under reduced pressure to remove ethyl acetate. Free DTXL was removed by using a tangential flow ultrafiltration system (Pall Filter (Beijing) Corporation).

For producing fluorescently labeled nanoparticles, mPEG-Dlinkₘ-PDLLA was taken as an example, and the production method was as follows. mPEG₁₁₃-Dlinkₘ-PDLLA₁₄₂ and PCL-RhoB were simultaneously dissolved in ethyl acetate in a mass ratio of 100:3. 200 μL (10 mg) of a stock solution of the polymer described above was taken, 1 mL of ultrapure water was added thereto, and ultrasonication was then performed for 1 min (0° C., 130 W, operation for 4s and stop for 2s, and 60 s in total). 2 mL of ultrapure water was added to the system, and after transferring to a round-bottomed flask, evaporation was immediately performed under reduced pressure to remove ethyl acetate. Free PCL-RhoB was removed by using a tangential flow apparatus.

For producing siRNA-entrapping nanoparticles, mPEG-Dlinkₘ-PLGA was taken as an example, and the production method was as follows. 400 μL of a trichloromethane stock solution of mPEG113-Dlinkₘ-PLGA_{161/54} (62.5 mg/mL) was taken, and 100 μL of a stock solution of BHEM-Chol (10 mg/mL, trichloromethane) was added. 25 μL of a stock solution of PLK1 siRNA (8 mg/mL) was further added, and ultrasonication was performed for 1 min (0° C., 130 W, operation for 5 s and stop for 2 s, and 60 s in total). 5 mL of RNase-free water was further added to the system, and ultrasonication was performed for 1 mM again (0° C., 130 W, operation for 10 s and stop for 2 s, and 60 s in total). After transferring to a round-bottomed flask, evaporation was immediately performed under reduced pressure to remove trichloromethane.

### Example 10

### Degradation Measurement of Nanoparticles

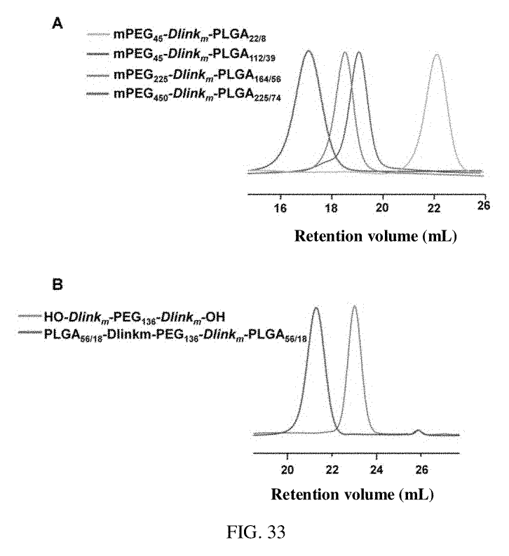

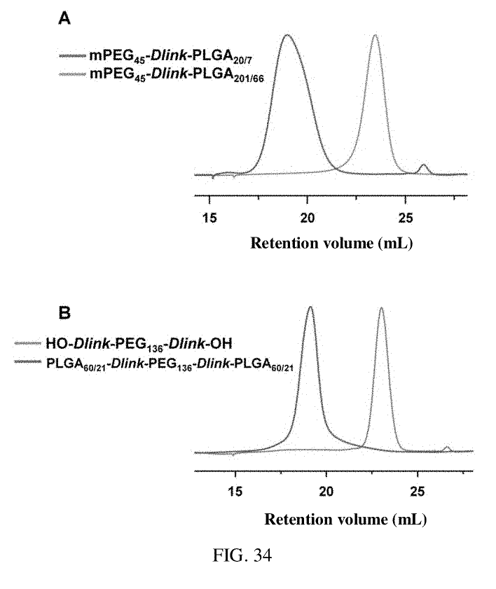

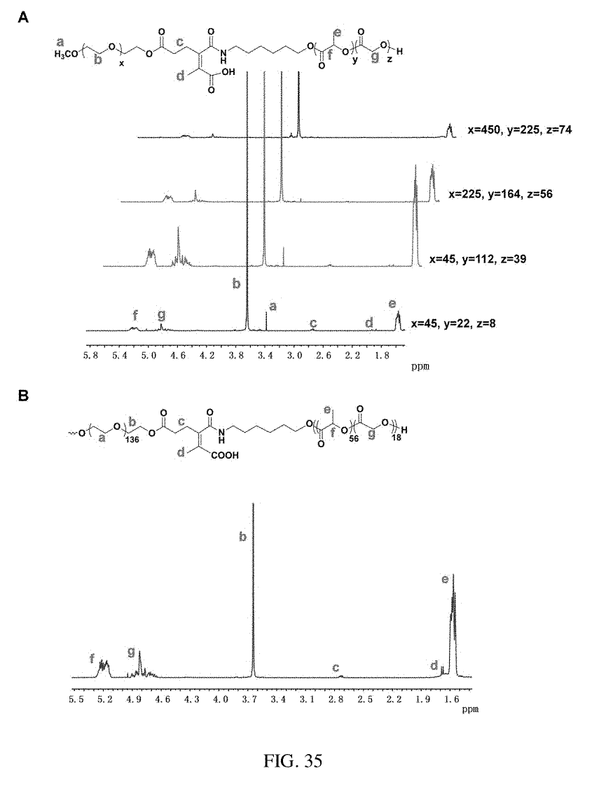

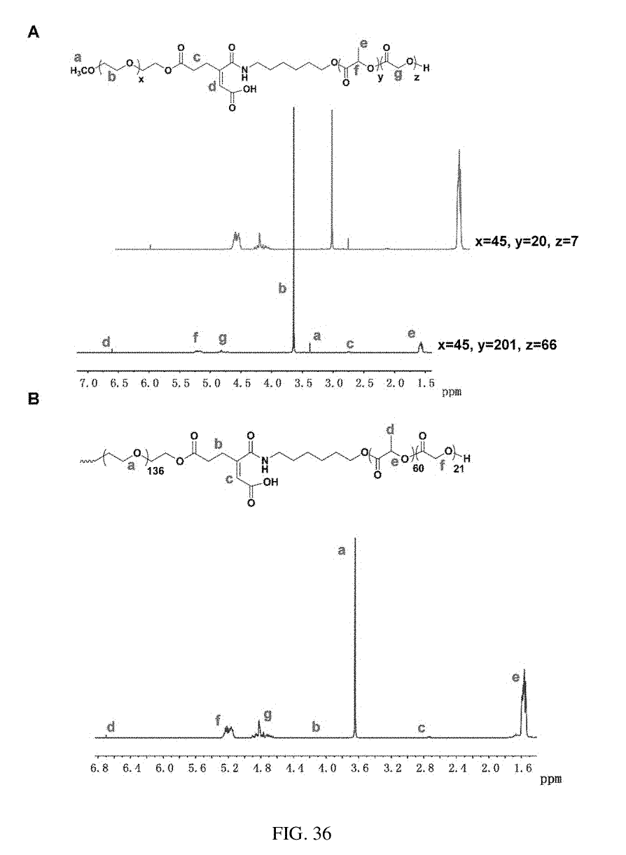

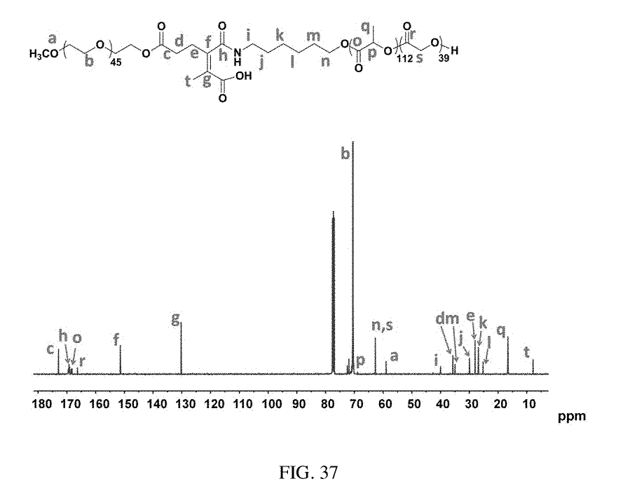

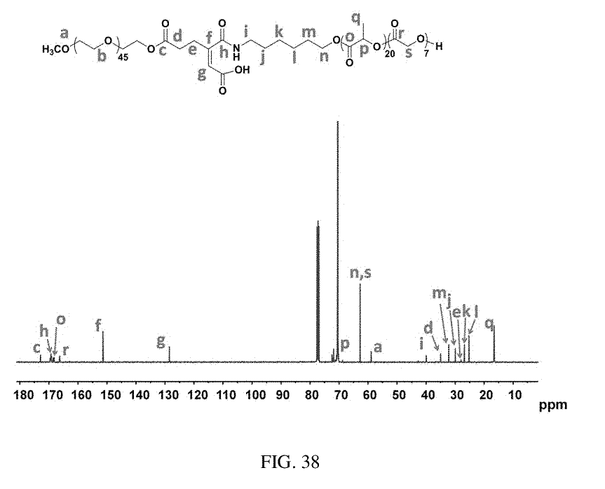

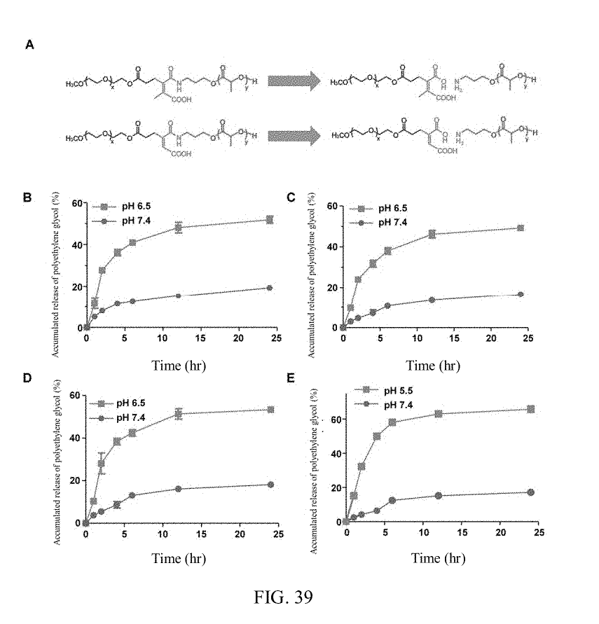

As shown in FIG. 39A, the amide bond in an acid-hydrolyzable chemical bond Dlinkₘ or Dlink would be degraded under a weakly acidic environment to generate two groups of homopolymers, which were polyethylene glycol and corresponding aliphatic polyester, respectively. In this Example, the acid sensitivity of the chemical bond Dlinkₘ or Dlink was detected under different pH conditions by the quantitative analysis of polyethylene glycol obtained by degradation. The production method of a single-emulsified nanoparticle in this Example was as shown in Example 9, and components, mPEG₁₁₃-Dlinkₘ-PDLLA₄₂, mPEG₁₁₃-DlinkₘPDLLA₇₁, mPEG₁₁₃-DlinkₘPDLLA₁₄₂, and mPEG₁₁₃-Dlink-PDLLA₁₄₀, were selected.

After nanoparticles were produced by using a single-emulsification method, the pH of a solution of particles was adjusted to 5.50, 6.50, and 7.40 by using a phosphate buffer (the concentration of the phosphate buffer was 20 mM), and the solution was treated at 37° C. and at a rotation speed of 60 rpm. The phosphate buffer solution containing 100 mg of nanoparticles was withdrawn at different time intervals. After 30 mM of 100000 g centrifugation, the liquid in the upper layer was lyophilized, and its release amount of PEG was detected by high-performance liquid chromatography. The results can be seen in FIG. 39.

As can be seen from FIG. 39, approximately more than 50% of PEG molecules in the Dlinkₘ bridged block polymer were released in 24 h under a condition of pH 6.5, while the release amount was less than 20% only under a control condition (pH 7.4). At the meanwhile, with respect to the Dlink bridged two-block polymer, more than 60% of PEG molecules were also responsively released under a condition of pH 5.5, which simulated an intracellular environment. It was demonstrated that PEG molecules could be relatively rapidly released under the stimulation of different tumor microenvironments by nanoparticles formed by assembling two types of polyethylene glycol aliphatic polyesters bridged by acid-hydrolyzable chemical bonds.

### Example 11

### Degradation of PEG by Nanoparticle Under Slightly Acidic Environment to Enhance Cell Uptake

In this Example, the behaviors of nanoparticles before and after the degradation of PEG under an acidic environment were studied by detecting the situation of the uptake of RhoB labeled nanoparticles by cells via flow cytometry. In this Example, mPEG₁₁₃-Dlinkₘ-PDLLA₁₄₂ and mPEG113-b-PDLLA₁₄₀ were used to produce fluorescently labeled nanoparticles. The production methods were as described in Example 9, and the particles were nominated as Dₘ-NP_{PDLLA} and NP_{PDLLA}.

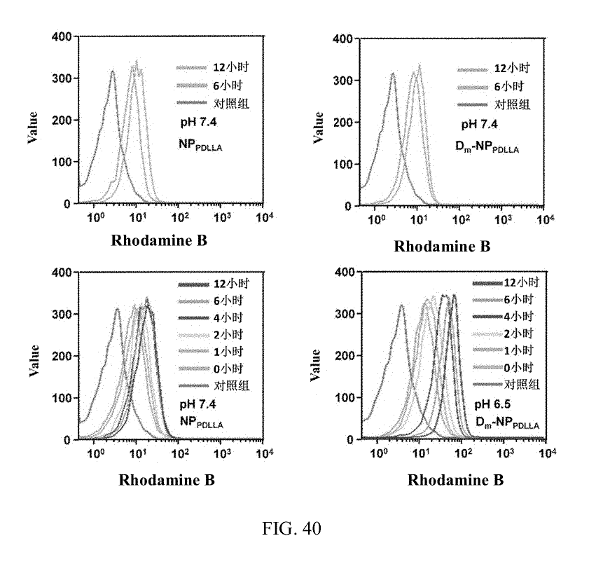

5×10⁴ MDA-MB-231 cells were plated in a 24-well plate, and 0.5 mL of a Dulbecco's Modified Eagle Medium (DMEM) complete medium was added. They were placed in a CO₂ incubator for culturing overnight, and a spent medium was suctioned. A fresh medium solution (treated for different times at pH 6.5 and 7.4, respectively) containing Dₘ-NP_{PDLLA} and NP_{PDLLA} was added to each well, and cultured for 2 h in a CO₂ incubator at 37° C. After completion of the experiment, cells were digested, washed with PBS twice, and resuspended with a 1% paraformaldehyde solution (200 μL). A flow cytometer (Becton Dickinson) was used for detection, and the results thereof were as shown in FIG. 40.

As can be seen from FIG. 40, with respect to NP_{PDLLA}, since there was no change in its nanoparticle components under two different pH conditions, there was no remarkable difference in its uptake behavior; whereas with respect to Dₘ-NP_{PDLLA}, the amount of its endocytosis was slightly increased under a condition of pH 7.4 condition compared to NP_{PDLLA}, but the cell uptake behavior was remarkably enhanced under a condition of pH 6.5. With reference to the degradation behaviors of nanoparticles in Example 10, it was considered that the degradation of the particle under a pH condition, which simulated a tumor environment, downregulated the density of PEG on the surface of the nanoparticle, and the barrier to the uptake of particles by PEG was solved.

### Example 12

### Situation of Circulation In Vivo on Single-Emulsified Nanoparticles

In this Example, the situation of the circulation of nanoparticles in the blood of mice was detected by high-performance liquid chromatography to investigate the blood circulation performances of nanoparticles formed by assembling a block copolymer bridged by an acid-hydrolyzable chemical bond and nanoparticles formed by assembling a block copolymer bridged by a non-acid-hydrolyzable chemical bond. The production methods of fluorescently labeled particles were as described in Example 9. In this Example, mPEG₁₁₃-Dlinkₘ-PDLLA₁₄₂ and mPEG₁₁₃-b-PDLLA₁₄₀ were selected as polymeric components. The nanoparticles produced were denoted by Dₘ-NP_{PDLLA} and NP_{PDLLA}.

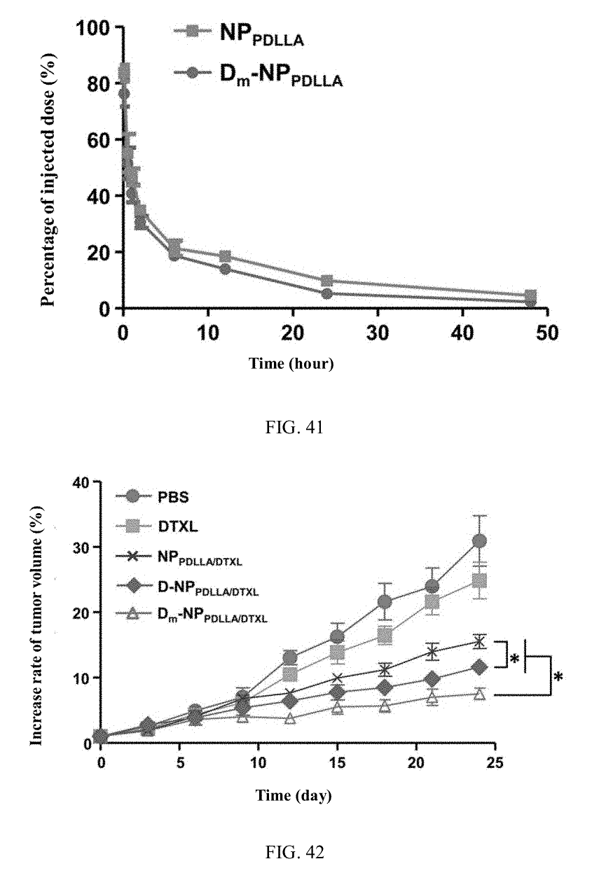

This Example was conducted in an ICR mouse. Dₘ-NP_{PDLLA} and NP_{PDLLA} were first injected through the tail vein, and each administration dosage of RhoB was 60 μg. At different time points after injection, blood was sampled through the venous plexus of the ocular fundus. After the addition of heparin sodium, the blood samples obtained were centrifuged at 10000 rpm for 5 min to obtain plasma. The plasma was extracted by an organic solvent and detected by high-performance liquid chromatography to analyze the content of RhoB therein. The results can be seen in FIG. 41. As can be seen from the figure, the situations of circulation in blood of two nanoparticles, Dₘ-NP_{PDLLA} and NP_{PDLLA}, were substantially the same, and there was no significant difference. It was demonstrated that the PEG layer could protect nanoparticles in the process of circulation in vivo and elongate the circulation time of blood.

### Example 13

### Inhibition of Breast Cancer Growth by Nanoparticles Carrying Chemotherapeutic Drugs

In this Example, a MDA-MB-231 in situ breast cancer mouse tumor model was used. The specific process of establishing the model was as follows. MDA-MB-231 cells were cultured in a DMEM complete medium. The cells were cultured with a serum-free DMEM 6 hours before the model was established, and digested with trypsin. The cells were collected by centrifugation at 1000 rpm, and resuspended with PBS to allow the density of the cells to be up to 2×10⁷cells/mL. 100 μL of the cell suspension was injected to the second right breast of a nude mouse. In this Example, mPEG113-b-PDLLA_{72,} mPEG113-Dlinkₘ-PDLLA_{70,} and mPEG₁₁₃-Dlink-PDLLA₇₅ were used to produce drug loaded nanoparticles. The production methods were as described in Example 9, and the particles were nominated as NP_{PDLLA/DTXL}, Dₘ-NP_{PDLLA/DTXL}, and D-NP_{PDLLA/DTXL}.

Nude mice, which were injected with breast cancer cells in situ, were bred in a SPF-level animal room for about 7 days, and visible tumors may be formed. The volume of the tumor was calculated according to the equation: V=0.5*a*b*b, wherein a refers to a longer diameter of the tumor and b refers to a shorter diameter of the tumor. When the tumor volume of the nude mouse reaches about 60 mm³, treatment was performed. 20 g nude mice inoculated with MDA-MB-231 tumor were divided into 5 groups according to the following manners of treatment, and each group has 5 nude mice. 200 μL of PBS, 200 μL of a PBS solution dissolved with 70 μg of Taxotere®, 200 4, of a PBS solution of NP_{PDLLA/DTXL,} 200 μL of Dₘ-NP_{PDLLA/DTXL}, and 200 μL of a PBS solution of D-NP_{PDLLA/DTXL} were used, respectively, wherein the amount of DTXL entrapped by the nanoparticle was 70 μg. A treatment period was 7 days and 3 administrations were performed. The tumor volume was measured every 3 days. The situation of tumor growth was shown in FIG. 42. The longitudinal coordinate in the figure was a ratio of the tumor volume obtained by measurement to the tumor volume on the first day. As can be seen from the figure, PBS and a low dosage of DTXL had no inhibitory effect on tumor growth. Compared to non-degradable NP_{PDLLA/DTXL} formed by assembling polyethylene glycol-polylactide, drug loaded nanomicelles, Dₘ-NP_{PDLLA/DTXL} and D-NP_{PDLLA/DTXL}, had stronger effects in inhibiting MDA-MB-231 breast cancer.

### Example 14

### siRNA Release Measurement of Nanoparticles Produced from Dlinkₘ Bridged Polyethylene Glycol-Dlinkₘ-Polylactide Copolymer Under Different pH Conditions

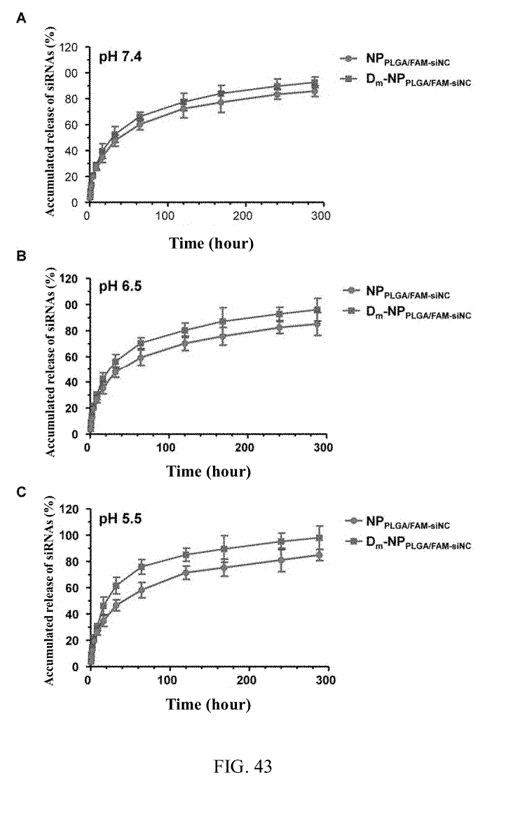

In this Example, mPEG113-Dlinkₘ-PLGA_{161/54} and mPEG113-b-PLGA_{165/56} were selected to produce nanoparticles carrying FAM-siNC by a double-emulsification method (Example 9) with the aid of BHEM-Chol. They were nominated as Dₘ-NP_{PLGA/FAM-siNC} and NP_{PLGA/FAM-siNC}, respectively. A solution of nanoparticles was diluted to 5 mL (10 mg/mL) with buffer solutions having pHs of 5.50, 6.50, and 7.40, respectively, and cultured at 37° C. and 60 rpm. At different time intervals, 100 μL of a solution of nanoparticles was taken, and centrifuged for 2 h (20000 g). The content of FAM-siNC in the supernatant was detected by high-performance liquid chromatography. The results can be seen in FIG. 43.

As can be seen from FIG. 43, siRNA release behaviors of two nanoparticles, Dₘ-NP_{PLGA/FAM-siNC} and NP_{PLGA/FAM-siNC}, were substantially the same under the three conditions of pH 7.4. Under slightly acid environments (pH 6.5 and pH 5.5), the siRNA release amounts of the two nanoparticles were 40%-60%, and the siRNA release of Dₘ-NP_{PLGA/FAM-siNC} was slightly faster that of NP_{PLGA/FAM-siNC}. It was demonstrated that the nanoparticle produced from a Dlinkₘ bridged polyethylene glycol-Dlinkₘ-poly(lactic-co-glycolic acid) copolymer and the nanoparticle formed by assembling a polyethylene glycol aliphatic polyester bridged by a non-acid-hydrolyzable chemical bond had similar release behaviors of siRNAs under various pH conditions inside and outside cells. Additionally, half of siRNAs were released by 24 h, and it was favorable to rapid silencing of gene expression in cells.

### Example 15

### Enhancement of the Cell Uptake by Polyethylene Glycol-Aliphatic Polyester Nanoparticles Under Slightly Acidic Environment

In this Example, the situation of the uptake of nanoparticles entrapping Cy5-siNC by cells was detected and observed by semi-quantitative flow cytometry, quantitative high-performance liquid chromatography, and qualitative laser confocal microscopy, respectively. In this Example, mPEG113-Dlinkₘ-PLGA_{161/54} and mPEG113-b-PLGA_{165/56} were selected to produce nanoparticles carrying Cy5-siNC by a double-emulsification method with the aid of BHEM-Chol. They were nominated as Dₘ-NP_{PLGA/Cy5-siNC} and NP_{PLGA/Cy5-siNC}, respectively.

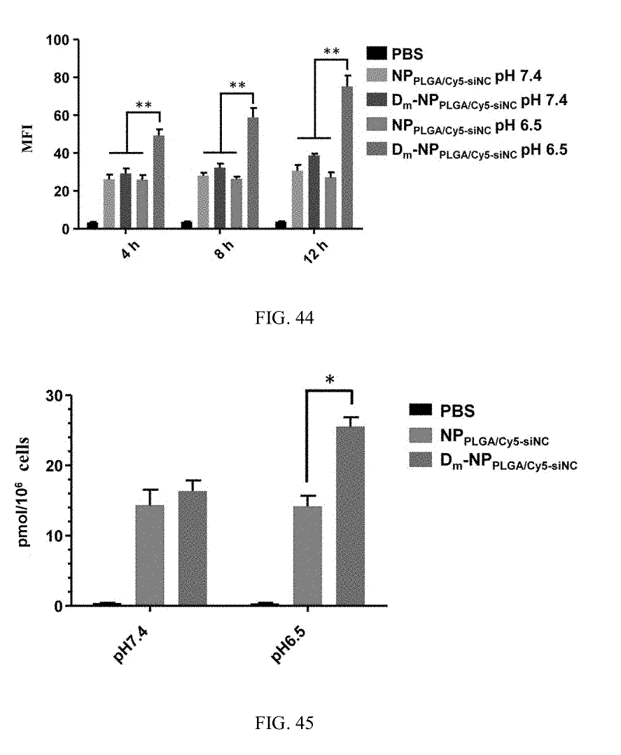

The situation of the uptake of nanoparticles by cells under an acidic environment was first semi-quantitatively detected by flow cytometry. 5×10⁴ MDA-MB-231 cells were plated in a 24-well plate, and 0.5 mL of a DMEM complete medium was added. They were placed in a CO₂ incubator for culturing overnight. A spent medium was suctioned, and a fresh medium solution (treated for different times at pH 6.5 and 7.4, respectively) containing Dₘ-NP_{PLGA/Cy5-siNC} and NP_{PLGA/Cy5-siNC} was added to each well, and cultured for 4 h in a CO₂ incubator at 37° C. After completion of the experiment, cells were digested, washed with PBS twice, and resuspended with a 4% paraformaldehyde solution (200 μL). A flow cytometer (Becton Dickinson) was used for detection, and the results thereof were as shown in FIG. 44. As can be seen from FIG. 44, with respect to NP_{PLGA/Cy5-siNC}, since there was no change in its nanoparticle components under two different pH conditions, there was no remarkable difference in its uptake behavior; whereas with respect to Dₘ-NP_{PLGA/Cy5-siNC}, the amount of its endocytosis was slightly increased under a condition of pH 7.4 condition compared to NP_{PLGA/Cy5-siNC}, but the cell uptake behavior was remarkably enhanced under a condition of pH 6.5. It may be considered that the degradation of the particle under a pH condition, which simulated a tumor environment, downregulated the density of PEG on the surface of the nanoparticle, and the uptake of nanoparticles carrying siRNAs by cells was enhanced.

The situations of the uptake of nanoparticles by cells before and after the degradation of PEG under an acidic environment were quantitatively detected by high-performance liquid chromatography. 2×10⁵ MDA-MB-231 cells were plated in a 6-well plate, and 2 mL of a DMEM complete medium was added. They were placed in a CO₂ incubator for culturing overnight, and a spent medium was suctioned. A fresh medium solution (treated at pH 6.5 and 7.4, respectively) containing Dₘ-NP_{PLGA/Cy5-siNC} and NP_{PLGA/Cy5-siNC} was added to each well, and cultured for 4 h in a CO₂ incubator at 37° C. After completion of the experiment, cells were digested, washed with PBS twice, and then lysed. The content of Cy5-siNC intracellularly uptaken was detected by high-performance liquid chromatography, and the results thereof were as shown in FIG. 45. As can be seen from FIG. 45, with respect to NP_{PLGA/Cy5-siNC}, the amounts of endocytosis of Cy5-siNC per million of cells were both about 15 pmol under conditions of pH 7.4 and pH 6.5; whereas with respect to Dₘ-NP_{PLGA/Cy5-siNC}, after treatment under a condition of pH 6.5, its amount of endocytosis of Cy5-siNC per million of cells was increased from 16 pmol under a condition of pH 7.4 to 25 pmol. This result also demonstrated that the nanoparticle produced from a Dlinkₘ bridged polyethylene glycol-Dlinkₘ-polylactide copolymer under a simulated acidic environment of tumor could enhance the uptake by cells.

The situations of the uptake of nanoparticles by cells before and after the degradation of PEG under an acidic environment were qualitatively detected by laser confocal microscopy. Coverslips were placed in a 24-well plate, 5×10⁴ MDA-MB-231 cells were plated, and 0.5 mL of a DMEM complete medium was added. They were placed in a CO₂ incubator for culturing overnight, and a spent medium was suctioned. A fresh medium solution (treated for different times at pH 6.5 and 7.4, respectively) containing Dₘ-NP_{PLGA/Cy5-siNC} and NP_{PLGA/Cy5-siNC} was added to each well, and cultured for 4 h in a CO₂ incubator at 37° C. After completion of the experiment, cells were fixed with a 4% paraformaldehyde solution. After 0.1% Triton X-100 penetration, the cytoskeleton was labeled by Alexa Fluor 488 and the cell nucleus was labeled by DAPI. A laser confocal microscope (Zeiss LSM 710) was used for observation, and the results thereof were as shown in FIG. 46. As can be seen from FIG. 46, the fluorescence signal of Cy5 in cells in the Dₘ-NP_{PLGA/Cy5-siNC} group was remarkably stronger that of NP_{PLGA/Cy5-siNC}. It was indirectly demonstrated that after the degradation of PEG, the particle could enhance the uptake of the particle by cells under a simulated acidic environment of tumor. Additionally, two intracellular particles were uniformly distributed in the cell plasma, and there was no remarkable difference in intracellular localization.

### Example 16

### Inhibition of mRNA Expression of PLK1 Gene in Breast Cancer Cell by Nanoparticles Entrapping Small Interfering RNAs Produced from a Dlinkₘ Bridged Polyethylene Glycol-Dlinkₘ-Poly(Lactic-co-Glycolic Acid) Copolymer Under Slightly Acidic Environment

In this Example, the effect of the uptake of nanoparticles on the PLK1 expression level under an acidic environment before and after the degradation of PEG was investigated by detecting the change in the PLK1 mRNA expression level after nanoparticles entrapping PLK1 siRNAs were uptaken by cells via a quantitative polymerase chain reaction (RT-PCR). The production methods of nanoparticles entrapping PLK1 siRNAs were as described in Example 9. The components selected were mPEG₁₁₃-Dlinkₘ-PLGA_{161/54} and mPEG₁₁₃-b-PLGA_{165/56.} The particles produced were denoted by Dₘ-NP_{PLGA/siPLK1} and NP_{PLGA/siPLK1}.

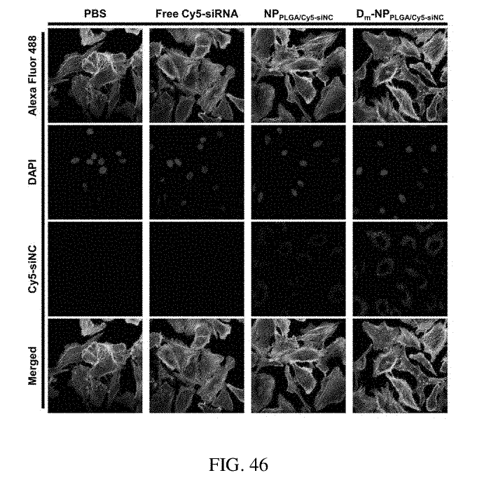

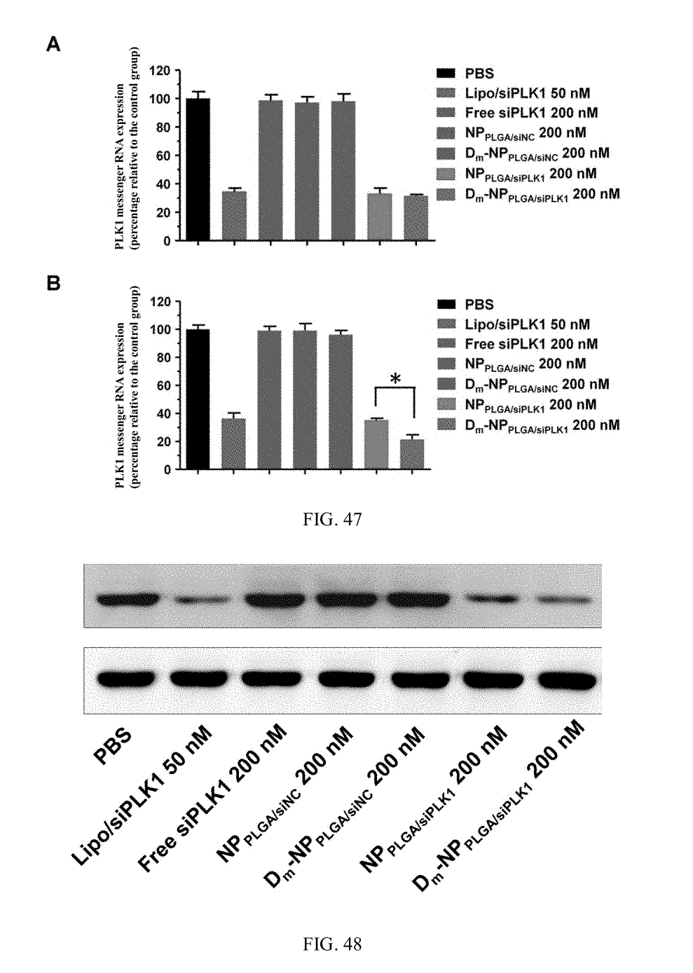

2×10⁵ MDA-MB-231 cells were plated in a 6-well plate, and 2 mL of a DMEM complete medium was added. They were placed in a CO₂ incubator for culturing overnight, and a spent medium was suctioned. Fresh medium solutions (the pH values of the media were set to be 6.5 and 7.4, respectively) containing Dₘ-NP_{PLGA/siPLK1}, NP_{PLGA/siPLK1}, nanoparticles Dₘ-NP_{PLGA/siNC} entrapping control siRNAs, and NP_{PLGA/siNC}, respectively were added to respective wells, and cultured for 6 h in a CO₂ incubator at 37° C. Media comprising nanoparticles were suctioned, and replaced with fresh media. Culture was continued for 24 h in a CO₂ incubator at 37° C. After completion of the experiment, cells were digested, and washed with PBS twice. The total RNA in the cell was then extracted by using RNAiso plus from Takara Corporation, the change in the PLK1 mRNA expression level was detected by a quantitative PCR method, and the results thereof were as shown in FIG. 47.

As can be seen from FIG. 47, under a condition of pH 7.4, since Dₘ-NP_{PLGA/siPLK1} nanoparticles were not subjected to degradation of a large amount of PEG and were not remarkably different from NP_{PLGA/siPLK1} in the surface PEG density, there was no significant difference in the level of inhibition of PLK1 mRNA expression in cells between Dₘ-NP_{PLGA/siPLKl} and NP_{PLGA/siPLK1}; whereas under a condition of pH 6.5, since Dₘ-NP_{PLGA/siPLK1} nanoparticles were subjected to the degradation of PEG under a slightly acidic environment to enhance cell uptake, the inhibition of PLK1 mRNA expression in cells by Dₘ-NP_{PLGA/siPLK1} was stronger compared to NP_{PLGA/siPLK1} and the expression of PLK1 mRNA was only 20% compared to a control group.

### Example 17

### Inhibition of Protein Expression of PLK1 Gene in Tumor Cells of Breast Cancer by Nanoparticles Entrapping Small Interfering RNAs Produced from a Dlinkₘ Bridged Polyethylene Glycol-Dlinkₘ-Poly(Lactic-co-Glycolic Acid) Copolymer Under Slightly Acidic Environment

In this Example, the effect of the uptake of nanoparticles on the PLK1 expression level under an acidic environment before and after the degradation of PEG was investigated by detecting the change in the PLK1 protein expression level after nanoparticles entrapping PLK1 siRNAs were uptaken by cells via a Western blot method. The production methods of nanoparticles entrapping PLK1 siRNAs were as described in Example 9. The components selected were mPEG₁₁₃-Dlinkₘ-PLGA_{161/54} and mPEG₁₁₃-b-PLGA_{165/56}. The particles produced were denoted by Dₘ-NP_{PLGA/siPLK1} and NP_{PLGA/siPLKl}.

2×10⁵ MDA-MB-231 cells were plated in a 6-well plate, and 2 mL of a DMEM complete medium was added. They were placed in a CO₂ incubator for culturing overnight. A spent medium was suctioned. Fresh medium solutions (the pH values of the media were set to be 6.5) containing Dₘ-NP_{PLGA/siPLK1}, NP_{PLGA/siPLK1}, nanoparticles Dₘ-NP_{PLGA/siNC} entrapping control siRNAs, and NP_{PLGA/siNC}, respectively were added to respective wells, and cultured for 6 h in a CO₂ incubator at 37° C. Media containing nanoparticles were suctioned, and replaced with fresh media. Culture was continued for 48 h in a CO₂ incubator at 37° C. After completion of the experiment, cells were digested, and washed with PBS twice. The total protein in the cell was then extracted by using NP40 protein lysis buffer from Beyotime Corporation. The change in the PLK1 protein expression level was detected by a Western blot method, and the results thereof were as shown in FIG. 48.

As can be seen from FIG. 48, the results under a condition of pH 6.5 were consistent with the experimental results of RT-PCR in Example 16. Since the degradation of Dₘ-NP_{PLGA/siPLK1} occurred to enhance cell uptake and intracellular PLK1gene silencing, the downregulation of PLK1 protein expression in cells by Dₘ-NP_{PLGA/siPLK1} was stronger compared to NP_{PLGA/siPLK1}.

### Example 18

### Inhibition of Breast Cancer Cell Proliferation by Nanoparticles Entrapping Small Interfering RNAs Produced from a Dlinkₘ Bridged Polyethylene Glycol-Dlinkₘ-Poly(Lactic-co-Glycolic Acid) Copolymer Under Slightly Acidic Environment

In this Example, the effect of the uptake of nanoparticles on cell proliferation under an acidic environment before and after the degradation of PEG was investigated by detecting the change in the cell viability after nanoparticles entrapping PLK1 siRNAs were uptaken by cells via a MTT method. The production methods of nanoparticles entrapping PLK1 siRNAs were as described in Example 9. The components selected were mPEG₁₁₃-Dlinkₘ-PLGA_{161/54} and mPEG113-b-PLGA_{16556.} The particles produced were denoted by Dₘ-NP_{PLGA/siPLK1} and NP_{PLGA/siPLK1}.

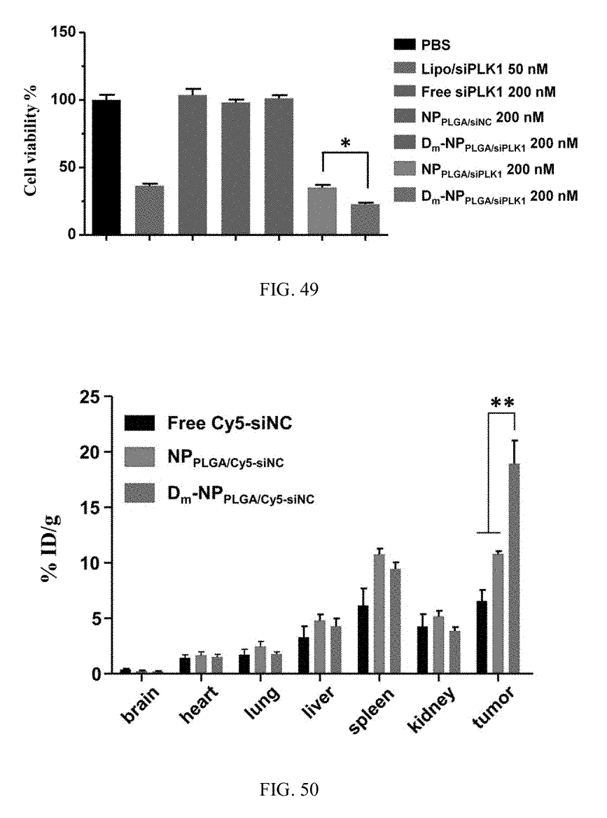

5×10³ MDA-MB-231 cells were plated in a 96-well plate, and 0.1 mL of a DMEM complete medium was added. They were placed in a CO₂ incubator for culturing overnight. A spent medium was suctioned. Fresh medium solutions (the pH values of the media were set to be 6.5) containing Dₘ-NP_{PLGA/siPLK1}, NP_{PLGA/siPLK1}, nanoparticles Dₘ-NP_{PLGA/siNC} entrapping control siRNAs, and NP_{PLGA/siNC}, respectively were added to respective wells, and cultured for 6 h in a CO₂ incubator at 37° C. Media containing nanoparticles were suctioned, and replaced with fresh media. Culture was continued for 72 h in a CO₂ incubator at 37° C. After completion of the experiment, 25 4, of 5 mg/mL thiazolyl blue was added to each well. After culturing for 2 h in a CO₂ incubator at 37° C., 100 μL of a cell lysis buffer was added to each well, and incubated in dark for 4 h at 37° C. The detection was performed with a microplate reader (Bio-rad), and the analytical results thereof were as shown in FIG. 49.

As can be seen from FIG. 49, under a condition of pH 6.5, since Dₘ-NP_{PLGA/siPLK1} can better induce PLK1 gene silencing, leading to the reduction of the capability of cell proliferation, the inhibitory capacity of Dₘ-NP_{PLGA/siPLK1} in cell proliferation was stronger compared to NP_{PLGA/siPLK1}.

### Example 19

### Distribution In Vivo of Nanoparticles Entrapping Small Interfering RNAs Produced from a Dlinkₘ Bridged Polyethylene Glycol-Dlinkₘ-Poly(Lactic-co-Glycolic Acid) Copolymer

In this Example, The distribution of nanoparticles in the organs within the body of a tumor-bearing mouse after siRNAs were carried was quantitatively detected by high-performance liquid chromatography. The production methods of particles entrapping Cy5-siNC were as described in Example 9. In this Example, mPEG₁₁₃-Dlinkₘ-PLGA_{161/54} and mPEG113-b-PLGA_{165/56} were selected as polymeric components. The nanoparticles produced were denoted by Dₘ-NP_{PLGA/Cy5-siNC} and NP_{PLGA/Cy5-siNC. In this Example, a MDA-MB-}231 in situ breast cancer mouse tumor model was used. The specific process of establishing the model was as described in Example 13.

Dₘ-NP_{PLGA/Cy5-siNC} and NP_{PLGA/Cy5-siNC} were injected through the tail vein, and the administration dosage of Cy5-siNC was 0.5 OD/injection. By 24 h after injection, the mouse was sacrificed. Organs of the mouse were taken, Cy5-siRNAs in tissues were extracted, and the content of siRNAs in the organs was detected by high-performance liquid chromatography. The results can be seen in FIG. 50. As can be seen from FIG. 50, two nanoparticles, Dₘ-NP_{PLGA/Cy5-siNC} and NP_{PLGA/Cy5-siNC}, were enriched to different extents in various organs, in which there were lessenrichments in organs such as brain, heart, lungs, and the like while there were more enrichments in organs related to metabolism and clearance mechanisms in vivo such as liver, kidneys, spleen, and the like. The enrichments of these two nanoparticles were not remarkably different in organs except tumors. At a tumor site, the enrichment of Dₘ-NP_{PLGA/Cy5-siNC} is remarkably higher than that of NP_{PLGA/Cy5-siNC}, demonstrating that Dₘ-NP_{PLGA/Cy5-siNC} enhanced the tumor cell uptake in an acidic microenvironment of tumor after the degradation of PEG so as to be capable of increasing the enrichment of siRNAs at the tumor site.

### Example 20

### Inhibition of Tumor Growth of Breast Cancer by Nanoparticles Entrapping Small Interfering RNAs Produced from a Dlinkₘ Bridged Polyethylene Glycol-Dlinkₘ-Poly(Lactic-co-Glycolic Acid) Copolymer

In this Example, a MDA-MB-231 in situ breast cancer mouse tumor model was used. The specific process of establishing the model was as described in Example 13. In this Example, mPEG113-Dlinkₘ-PLGA_{161/54} and mPEG₁₁₃-b-PLGA_{165/56} were used to produce nanoparticles carrying PLK1 siRNAs. The production methods were as described in Example 9, and the particles were nominated as Dₘ-NP_{PLGA/siPLK1} and NP_{PLGA/siPLK1}.

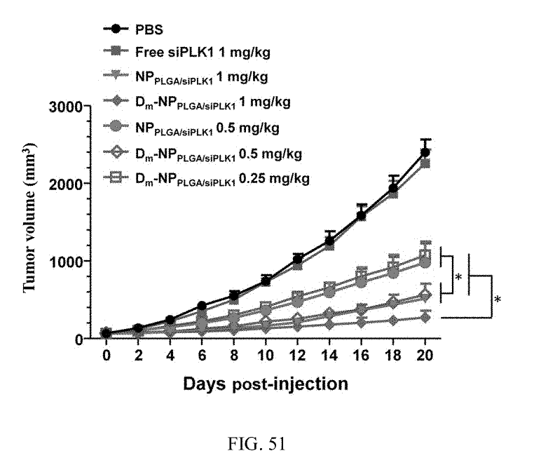

Nude mice, which were injected with breast cancer cells in situ, were bred in a SPF-level animal room for about 7 days, and visible tumors may be formed. The volume of the tumor was calculated according to the equation: V=0.5*a*b*b, wherein a refers to a longer diameter of the tumor and b refers to a shorter diameter of the tumor. When the tumor volume of the nude mouse reaches about 60 mm³, treatment was performed. 20 g nude mice inoculated with MDA-MB-231 tumor were divided into 7 groups according to the following manners of treatment, and each group has 5 nude mice. According to the administration dosage of PLK1 siRNAs used as a standard for calculation, experimental groups were provided as follows: a PBS group, Free siPLK1 1 mg/kg, NP_{PLGA/siPLK}1 1 mg/kg, Dₘ-NP_{PLGA/siPLK1} 1 mg/kg, NP_{PLGA/siPLK1} 0.5 mg/kg, DₘNP_{PLGA/siPLKl} 1 0.5 mg/kg, and Dₘ-NP_{PLGA/siPLK}1 0.25 mg/kg. The above drugs were formulated with 400 μL of PBS for administration. A treatment period was 2 days and 10 administrations were performed. The tumor volume was measured every 2 days. The change in the tumor volume was shown in FIG. 51, and the longitudinal coordinate in the figure was the tumor volume obtained by measurement. As can be seen from the figure, Dₘ-NP_{PLGA/siPLK1} at administration dosages of 1 mg/kg and 0.5 mg/kg has an inhibitory effect on tumor growth remarkably superior to NP_{PLGA/siPLK1} at the same dosage.

Although this invention is set forth for the purpose of illustration and description, exhaustion and limitation are not intended. Various modifications and variations are apparent to those of skill in the art. The technical solutions are selected and described in order to explain fundamental and practical applications, and the person skilled in the art will be understood that various embodiments of this invention having various modifications are suitable for intended particular applications.

1. A bridged polyethylene glycol-aliphatic polyester block copolymer, having the following general structural formula III: 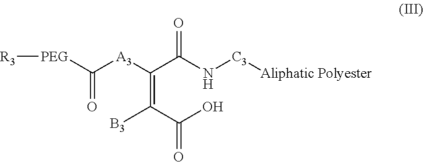
wherein A₃ is selected from C_{g}Hₕ, and g and h are integers, 0≤g≤4, and 0≤h≤10; B₃ is a methyl group or absent; C₃ is selected from CᵢHⱼ, and i and j are integers, 1≤i≤20, and 2≤j≤42; R₃ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue, and aliphatic polyester represents an aliphatic polyester residue.

2. The bridged polyethylene glycol-aliphatic polyester block copolymer according to claim 1, wherein A₃ is absent or an alkylene group having a carbon atom number of 1-4.

3. The bridged polyethylene glycol-aliphatic polyester block copolymer according to claim 1, wherein C₃ is an alkylene group having a carbon atom number of 1-20, more preferably an alkylene group having a carbon atom number of 1-6.

4. The bridged polyethylene glycol-aliphatic polyester block copolymer according to claim 1, wherein R₃ is an alkoxy group having a carbon atom number of 1-6.

5. The bridged polyethylene glycol-aliphatic polyester block copolymer according to claim 1, wherein the polyethylene glycol residue is represented by the following general formula: 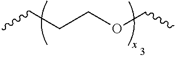
wherein x₃ is an integer and 1≤x₃≤500;
the aliphatic polyester residue is a residue of poly(ε-caprolactone), polylactide, or poly(lactic-co-glycolic acid).

6. The bridged polyethylene glycol-aliphatic polyester block copolymer according to claim 5, wherein the number-average molecular weight of the aliphatic polyester is 2000-20000; more preferably 5000-15000.

7. The bridged polyethylene glycol-aliphatic polyester block copolymer according to claim 6, wherein the ratio of repeating units of lactic acid to glycolic acid in the poly(lactic-co-glycolic acid) is 10-90/90-10, more preferably 20-80/80-20, and further preferably 75/25.

8. A production method of the bridged polyethylene glycol-aliphatic polyester block copolymer of any one of claims 1-7, comprising: performing ring opening polymerization reaction of an aliphatic polyester monomer by using a maleamidic acid derivative modified polyethylene glycol as an initiator to obtain a bridged polyethylene glycol-aliphatic polyester block copolymer; or performing macromolecular coupling reaction between a maleamidic acid derivative modified polyethylene glycol and an aliphatic polyester having an amino terminal group to obtain a bridged polyethylene glycol-aliphatic polyester block copolymer.

9. The production method of the bridged polyethylene glycol-aliphatic polyester block copolymer according to claim 8, wherein the solvent is dichloromethane.

10. A maleamidic acid derivative modified polyethylene glycol, having the following general structural formula II: 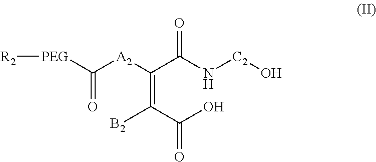
wherein A₂ is selected from C_{c}H_{d}, and c and d are integers, 0≤c≤4, and 0≤d≤10; B₂ is a methyl group or absent; C₂ is selected from CₑH_{f}, and e and f are integers, 1≤e≤20, and, 2≤f≤42; R₂ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue.

11. A production method of the maleamidic acid derivative modified polyethylene glycol according to claim 10, comprising mixing an amino alcohol with a polyethylene glycol containing a maleic anhydride group at the terminal, and forming an amide bond by performing ring opening reaction using a primary amine group in the amino alcohol and the maleic anhydride group to obtain a maleamidic acid derivative modified polyethylene glycol.

12. A polyethylene glycol containing a maleic anhydride group at the terminal, having the following general structural formula I: 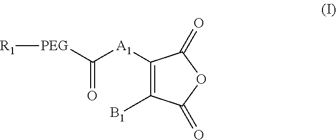
wherein A₁ is selected from CₐH_{b}, and a and b are integers, 0≤a≤4, and 0≤b≤10; B₁ is a methyl group or absent; R₁ is absent or an alkyl group, an alkoxy group, an aryl group, an aryloxy group, a halogen atom, or a substituted alkyl group, a substituted alkoxy group, a substituted aryl group, or a substituted aryloxy; and PEG represents a polyethylene glycol residue.

13. A production method of the polyethylene glycol containing a maleic anhydride group at the terminal according to claim 12, comprising subjecting a carboxy group in a carboxy-substituted maleic anhydride to acyl chlorination and then to reaction with a terminal hydroxy group of the polyethylene glycol.

14. A pharmaceutical carrier or nucleic acid carrier produced from the bridged polyethylene glycol-aliphatic polyester block copolymer of any one of claims 1-7.

15. A drug loaded nanoparticle or nucleic acid loaded nanoparticle produced from the pharmaceutical carrier or nucleic acid carrier of claim 14.

16. A method of treating tumors comprising administrating the pharmaceutical carrier or nucleic acid carrier produced from the maleamidic acid derivative modified polyethylene glycol of claim 10, the pharmaceutical carrier or nucleic acid carrier produced from the polyethylene glycol containing a maleic anhydride group at the terminal of claim 12, the pharmaceutical carrier or nucleic acid carrier of claim 14, and the drug loaded nanoparticle or nucleic acid loaded nanoparticle of claim 15 to a subject.
